(19) United States
(12) Patent Application Publication
(10) Pub. No.: US 2024/0108355 A1
(43) Pub. Date: Apr. 4, 2024

(54) CARTRIDGE SYSTEM, CARTRIDGE AND METHOD FOR LOADING CLIP UNIT

(71) Applicant: OLYMPUS MEDICAL SYSTEMS CORP., Tokyo (JP)

(72) Inventors: Kensuke Uesaka, Hino-shi (JP); Yoshihisa KANEKO, Hirosaki-shi (JP); Hiroshi MINAMI, Chofu-shi (JP)

(21) Appl. No.: 18/479,347

(22) Filed: Oct. 2, 2023

Related U.S. Application Data
(60) Provisional application No. 63/378,169, filed on Oct. 3, 2022.

(51) Int. Cl. A61B 17/122; A61B 17/128

(52) U.S. Cl. CPC A61B 17/1222; A61B 17/1285

(57) ABSTRACT

A cartridge system includes a clip unit including an arm configured to be opened and closed, and a case in which the clip unit has a storage area configured to move along a longitudinal direction, wherein the case includes an opening through which at least part of the arm is exposed when the clip unit stored in the storage area is positioned at a predetermined position.

### CROSS-REFERENCE TO RELATED APPLICATIONS

The present application claims priority based on U.S. Patent Provisional Application No. 63/378,169 provisionally filed in the United States on Oct. 3, 2022, the contents of which are incorporated herein by reference.

### TECHNICAL FIELD

The present disclosure relates to a cartridge system accommodating an endoscope clip unit, a cartridge accommodating the endoscope clip unit, and a method for loading the endoscope clip unit.

### BACKGROUND

In endoscopic treatment, a clip unit is used that can ligate a resected area after treatment to stop bleeding. The clip unit includes a clip for sandwiching the resected portion or the like, and a holding tube or the like for accommodating the clip and locking it in a closed state. The clip unit is introduced to the treatment position by a clip introduction device that can be inserted through a channel of the endoscope.

A dedicated fixture may be used when loading the clip unit into the clip introduction device. For example, Japanese Unexamined Patent Application, First Publication No. 2009-189704 (hereinafter referred to as Patent Document 1) describes a fixture provided with a through-window.

In the fixture described in Patent Document 1, if the clip moves inside the fixture, the operator may not be able to see the clip through the through-window.

### SUMMARY

In view of the above circumstances, the present disclosure provides a cartridge system, a cartridge, and a method of loading a clip unit that allow visual confirmation of the clip of the clip unit stored inside.

In order to solve the above problems, the present disclosure proposes the following means.

A cartridge system according to a first aspect of the present disclosure includes a clip unit including an arm that can be opened and closed, and a case in which the clip unit includes a storage area that can move along a longitudinal direction, wherein the case includes an opening through which at least part of the arm is exposed when the clip unit stored in the storage area is positioned at a predetermined position.

A cartridge system according to a second aspect of the present disclosure includes a clip unit including an arm that can be opened and closed, a case in which the clip unit includes a storage area that can move along a longitudinal direction, and a thread with one end tied to the arm.

According to the cartridge system, cartridge, and clip unit-loading method of the present disclosure, the clip of the clip unit stored inside can be visually recognized.

### BRIEF DESCRIPTION OF DRAWINGS

FIG. 1 is a perspective view of a clip introduction device.

FIG. 2 is a perspective view of a clip unit of a cartridge system according to a first embodiment.

FIG. 3 is a perspective view of the clip unit in which a holding member is transparently displayed.

FIG. 4 is a longitudinal sectional view of the holding member.

FIG. 5 is a perspective view of a connecting member of the clip unit.

FIG. 6 is a perspective view of a cartridge storing the clip unit.

FIG. 7 is a sectional view of the cartridge storing the clip unit.

FIG. 8 is a cross-sectional view of the cartridge before storing the clip unit.

FIG. 9 is a perspective view of a case of the cartridge in which the clip unit is not stored.

FIG. 10 is a perspective view showing an engaging portion of the case.

FIG. 11 is a front view of the case viewed from a direction of an arrow shown in FIG. 9.

FIG. 12 is a perspective view of a restricting member of the cartridge.

FIG. 13 is a plan view of the restricting member.

FIG. 14 is a diagram showing an intermediate layer member of the restricting member grasped by a pair of arms.

FIG. 15 is a diagram showing the cartridge in which the clip unit is stored.

FIG. 16 is a diagram showing the cartridge in which a thread is hooked on a pair of arms.

FIG. 17 is a diagram showing a method of loading the clip unit into the clip introduction device using the cartridge.

FIG. 18 is a diagram showing a method of loading the clip unit into the clip introduction device using the cartridge.

FIG. 19 is a diagram showing a method of loading the clip unit into the clip introduction device using the cartridge.

FIG. 20 is a diagram showing a method of loading the clip unit into the clip introduction device using the cartridge.

FIG. 21 is a diagram showing a method of loading the clip unit into the clip introduction device using the cartridge.

FIG. 22 is a diagram showing a method of loading the clip unit into the clip introduction device using the cartridge.

FIG. 23 is a diagram showing a method of loading the clip unit into the clip introduction device using the cartridge.

FIG. 24 is a diagram showing a method of loading the clip unit into the clip introduction device using the cartridge.

FIG. 25 is a diagram showing a method of loading the clip unit into the clip introduction device using the cartridge.

FIG. 26 is a diagram showing a method of loading the clip unit into the clip introduction device using the cartridge.

FIG. 27 is a diagram explaining a method of storing the clip unit in the cartridge.

FIG. 28 is a diagram explaining a method of storing the clip unit in the cartridge.

FIG. 29 is a diagram explaining a method of storing the clip unit in the cartridge.

FIG. 30 is a diagram explaining a method of storing the clip unit in the cartridge.

FIG. 31 shows a package containing the cartridge.

FIG. 32 shows a package containing the cartridge.

FIG. 33 shows a package containing the cartridge.

FIG. 34 shows a package containing the cartridge.

FIG. 35 shows a package containing a cartridge having no opening.

FIG. 36 shows a package containing a cartridge having no opening.

FIG. 37 is a view showing a modification of the clip of the clip unit.

FIG. 38 is a diagram showing another modification of the clip of the clip unit.

FIG. 39 is a view showing another modification of the clip of the clip unit.

FIG. 40 is a diagram showing a modification of the restricting member.

FIG. 41 is a view showing another modification of the restricting member.

FIG. 42 is a view showing another modification of the restricting member.

FIG. 43 is a diagram showing another modification of the restricting member.

FIG. 44 is a diagram showing a cartridge system according to a second embodiment.

FIG. 45 is a perspective view of a clip unit of the cartridge system.

FIG. 46 is a diagram showing an intermediate layer member of the restricting member of the cartridge system.

FIG. 47 is an enlarged view of a distal end of a second arm of the clip unit.

FIG. 48 is a diagram showing a pair of arms of the clip unit pulled to a proximal end side.

FIG. 49 is a diagram showing the pair of arms whose distal ends are in contact with the restricting member.

FIG. 50 is a diagram showing a modification of the pair of arms.

### EMBODIMENTS FOR CARRYING OUT THE DISCLOSURE

### First Embodiment

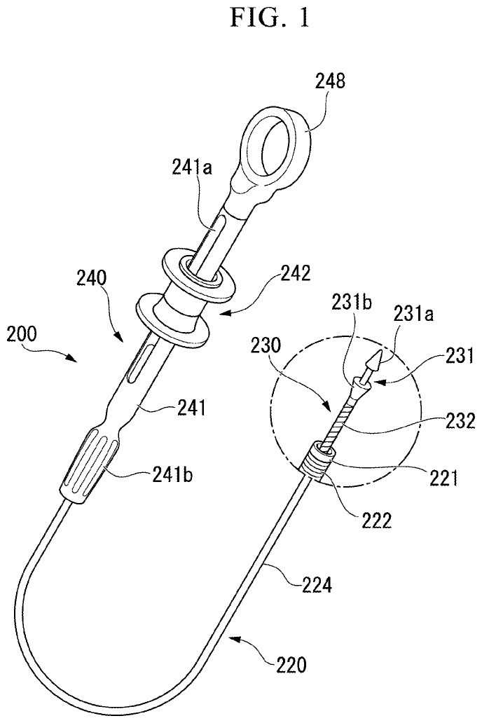

A cartridge system 100 according to a first embodiment of the present disclosure will be described with reference to FIGS. 1 to 36. The cartridge system 100 includes a clip unit 1 and a cartridge 5 housing the clip unit 1. The cartridge system 100 is a support system for easily loading the clip unit 1 into a clip introduction device 200.

### [Clip Introduction Device 200]

FIG. 1 is a perspective view of the clip introduction device 200.

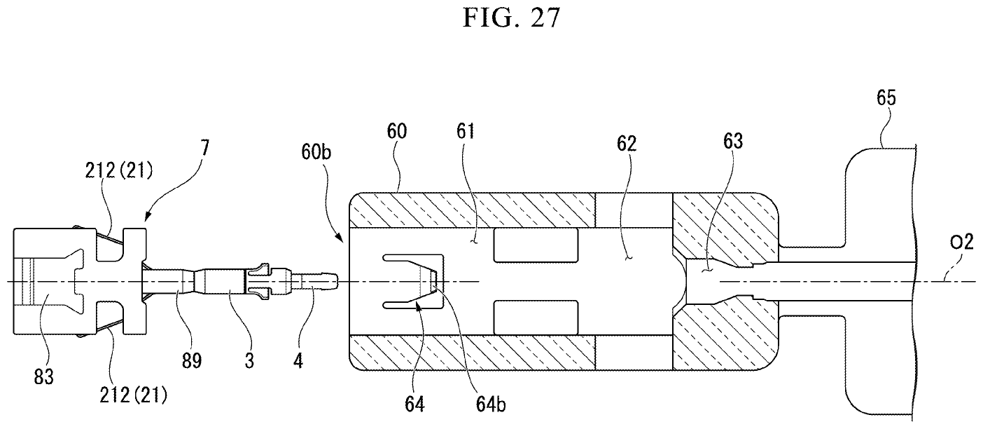

The clip introduction device (applicator) 200 includes a sheath 220, an operating wire 230, and an operation portion 240. The clip introduction device 200 is inserted through, for example, a treatment instrument insertion channel 310 of an endoscope 300 and used in combination with the endoscope 300 (FIG. 27). Therefore, the sheath 220 is formed sufficiently longer than the treatment instrument insertion channel 310 of the endoscope 300. The sheath 220 is flexible and curves according to the curvature of an insertion portion of the endoscope 300.

The sheath 220 includes a distal tip 221, a distal coil 222, and a proximal coil 224, and is formed in an elongated tubular shape as a whole. The distal coil 222 is arranged on the distal side of the sheath 220. The distal tip 221 is arranged at the distal end of the distal coil 222.

The operation wire (power transmission portion) 230 includes, as shown in FIG. 1, an arrowhead hook portion (connecting portion) 231 connected to the clip unit 1, and a wire 232 that operates the arrowhead hook portion 231.

The arrowhead hook portion 231 includes a substantially conical engaging portion 231a that engages with the clip unit 1, and a wire connecting portion 231b provided at the proximal end of the engaging portion 231a. The arrowhead hook portion 231 is made of, for example, a metal material such as stainless steel.

The wire 232 is inserted through the sheath 220 so as to be able to advance and retract. The distal end of the wire 232 is fixed to the proximal end of the wire connecting portion 231b by welding, for example.

The operation portion 240 includes an operation portion main body 241, a slider 242, and a thumb ring 248, as shown in FIG. 1. The operation portion main body 241 is injection-molded, for example, from a resin material. The operation portion main body 241 includes a slit portion 241a and a rotary grip 241b on the distal end side. The slit portion 241a supports the slider 242 so as to be able to advance and retract.

The slider 242 is attached so as to be able to move back and forth in the longitudinal axis direction of the operation portion main body 241, and the proximal end of the wire 232 is attached. As the slider 242 advances and retracts along the operation portion main body 241, the wire 232 advances and retracts with respect to the sheath 220, and the arrowhead hook portion 231 advances and retracts.

The thumb ring 248 is attached to the proximal end of the operation portion main body 241 so as to be rotatable around the longitudinal axis of the operation portion main body 241.

### [Clip Unit 1]

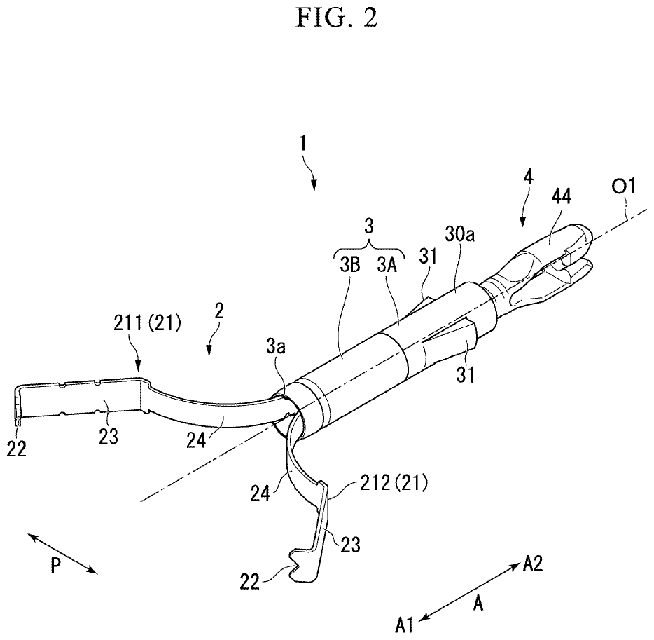

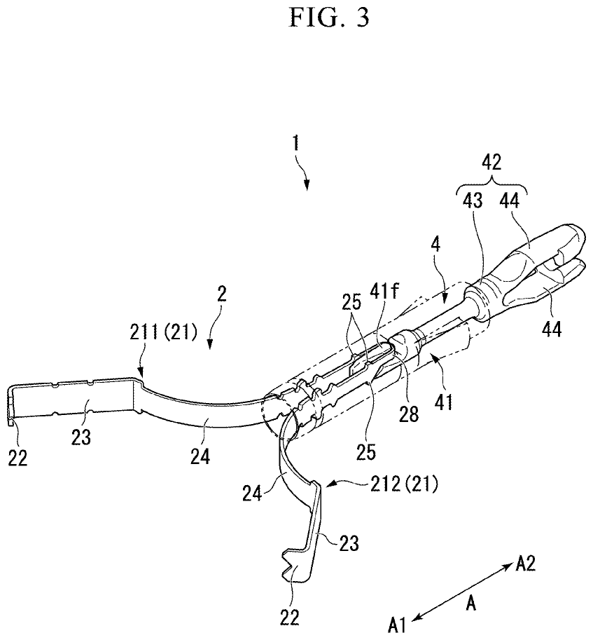

FIG. 2 is a perspective view of the clip unit 1. FIG. 3 is a perspective view of the clip unit 1 in which the holding member 3 is transparently displayed. The clip unit 1 includes a clip 2, a holding member 3 and a connecting member 4. In the following description, the side of the clip 2 in the longitudinal direction A of the clip unit 1 is referred to as the distal end side (distal side) A1 of the clip unit 1, and the side of the connecting member 4 is referred to as the proximal end side (proximal side) A2 of the clip unit 1.

The clip 2 is formed by bending a metal plate at the center. The clip 2 includes a pair of arms 21 that can be opened and closed, and a proximal end portion 28 that connects the pair of arms 21. The proximal end side A2 of the clip 2 is inserted into the internal space 38 of the holding member 3.

The pair of arms 21 includes a first arm 211 and a second arm 212. The first arm 211 and the second arm 212 are arranged on both sides of the central axis O1 in the longitudinal direction A of the clip unit 1. The clip 2 may include three or more arms.

The first arm 211 includes a tissue-grasping portion 22, a flat plate-like grasping portion 23, a sliding portion 24, and an engaging portion 25 from the distal side A1 to the proximal side A2. The tissue-grasping portion 22 is formed by bending the distal end of the first arm 211 inward. The sliding portion 24 is a portion that is elastically deformed when the pair of arms 21 are pulled into the holding member 3.

The second arm 212 includes the tissue-grasping portion 22, the flat plate-like grasping portion 23, the sliding portion 24, and the engaging portion 25 from the distal side A1 to the proximal side A2.

The tissue-grasping portion 22 of the first arm 211 and the tissue-grasping portion 22 of the second arm 212 are formed in an asymmetrical shape with respect to the central axis O1. Therefore, when attaching the clip 2 to the connecting member 4, the user can easily grasp the direction in which the clip 2 is to be attached to the connecting member 4. The tissue-grasping portion 22 of the first arm 211 and the tissue-grasping portion 22 of the second arm 212 may be formed in a shape symmetrical with respect to the central axis O1.

The first arm 211 and the second arm 212 may include a curve portion. The curve portion is curved toward to the central axis O1 at proximal side of the tissue-grasping portion 22.

The engaging portion 25 is a member that can be engaged with a tightening member 32 of the holding member 3. The distal end side A1 of the engaging portion 25 is formed with an obtuse slope with respect to the longitudinal direction A, and the proximal end side A2 is formed with an acute angle with respect to the longitudinal direction A.

The proximal end portion 28 connects the first arm 211 and the second arm 212. The first arm 211 and the second arm 212 connected by the proximal end portion 28 are provided so as to be openable and closable toward the distal end side A1. The proximal end portion 28 is bent to have a U-shape and is connected to a hook 41f of the connecting member 4. The proximal end portion 28 is biased so that the pair of arms 21 are in an open state. Therefore, the pair of arms 21 of the clip 2 have a self-expanding force in the opening/closing direction P.

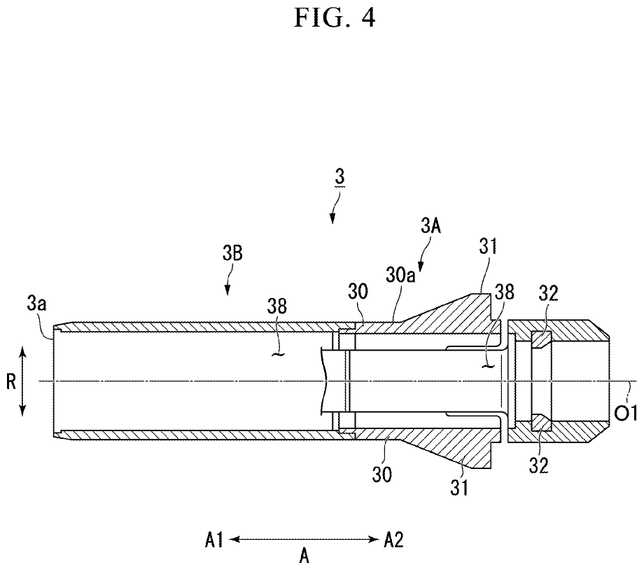

As shown in FIG. 4, the proximal end portion 28 and the hook 41f of the connecting member 4 are connected by inserting the hook 41f of the connecting member 4 into the proximal end portion 28 formed in a U shape.

FIG. 4 is a cross-sectional view of the holding member 3 in the longitudinal direction A.

The holding member (tubular member) 3 is a circular tubular member capable of storing at least part of the clip 2. The holding member 3 includes an internal space 38 in which the clip 2 advances and retracts in the longitudinal direction A. The holding member 3 can fix the clip 2 pulled into the internal space 38 in a closed state. The holding member 3 includes a holding tube 3A provided on the proximal side A2 and a holding tube 3B provided on the distal side A1.

The holding tube (second tubular member) 3A includes a holding tube main body 30 formed in a tubular shape, a protruding/retracting wing 31 and a tightening member 32.

The holding tube main body 30 is formed by injection molding a material softer than that of the clip 2 such as PPA (polyphthalamide), PA (polyamide), PEEK (polyetheretherketone), LCP (liquid crystal polymer), or other thermoplastic resin having appropriate elasticity. The hold down tube main body 30 may be made of metal instead of thermoplastic resin.

The protruding/retracting wings 31 are a pair of convex portions that protrudes/retracts against the outer peripheral surface 30a of the holding tube main body 30. The protruding/retracting wings 31 are provided on both sides of the central axis O1. The protruding/retracting wing 31 has a basic posture in which it projects outward in the radial direction R with respect to the outer peripheral surface 30a. The protruding/retracting wing 31 receives a force directed from the outside toward the inside in the radial direction R, so that it is in a retracted state in which it is retracted with respect to the outer peripheral surface 30a. By the above force being released, the protruding/retracting wing 31 returns from the collapsed state to the projected state.

The tightening member 32 is a ring-shaped member provided in the internal space 38 of the holding tube 3A. The tightening member 32 is made of metal. The tightening member 32 may be formed so as to be harder than the holding tube main body 30, and may be formed of thermoplastic resin instead of metal, for example.

The tightening member 32 is arranged so that the central axis of the tightening member 32 coincides with the central axis O1. The tightening member 32 is arranged on the proximal end side A2 with respect to the protruding/retracting wing 31. The tightening member 32 is incorporated into the holding tube main body 30 by, for example, insert molding. The tightening member 32 is arranged at a position protruding inward in the radial direction R from the inner peripheral surface of the holding tube main body 30.

The holding tube (first tubular member) 3B is a metallic cylindrical member. The holding tube 3B is press-fitted into the distal end of the holding tube 3A. The holding tube 3A and the holding tube 3B may be connected by heat welding, adhesion or screwing.

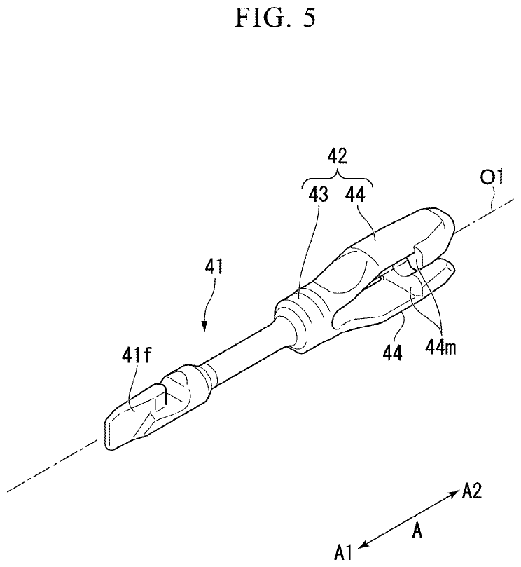

FIG. 5 is a perspective view of the connecting member 4.

The connecting member 4 is detachably connected to the proximal end portion 28 of the clip 2. In addition, the connecting member 4 is detachably connected to an arrowhead hook portion 231 passing through the sheath 220. That is, the connecting member 4 connects the clip 2 and the arrowhead hook portion 231. The connecting member 4 includes an insertion portion 41 inserted into the internal space 38 of the holding member 3 and a connecting portion 42 provided at the proximal end of the insertion portion 41.

The insertion portion 41 includes the hook 41f at its distal end. The hook 41f is a hook extending in a direction perpendicular to the central axis O1, and is formed in a substantially columnar rod shape. The proximal end portion 28 of the clip 2 is hooked on the hook 41f. The hook 41f breaks when a predetermined amount of breaking force is applied by pulling the proximal end portion 28 toward the proximal end side.

The connecting portion 42 is an engaging portion with which the arrowhead hook portion 231 of the clip introduction device 200 is engaged (connected). The connecting portion 42 includes a connecting portion main body 43 and an elastic arm portion 44.

The elastic arm portion 44 is provided at the proximal end of the connecting portion main body 43 and is bifurcated. The elastic arm portion 44 is elastically deformable with respect to the connecting portion main body 43 and can be opened and closed with respect to the connecting portion main body 43. The elastic arm portion 44 is formed with a notch portion 44m for grasping and housing the engaging portion 231a of the arrowhead hook portion 231. The notch portion 44m is formed in a shape that closely contacts the outer peripheral surface of the engaging portion 231a of the arrowhead hook portion 231.

Next, the action of the clip unit 1 will be described.

The proximal end portion 28 of the clip 2 is inserted into the inner space of the holding member 3 from the distal end opening 3a and connected to the connecting member 4. By pulling the proximal end portion 28 toward the proximal end side A2 of the holding member 3 by the connecting member 4, the pair of arms 21 is pulled into the holding member 3, and the pair of arms 21 gradually closes. When the pulling force of the proximal end portion 28 is released in this state, the self-expanding force of the pair of arms 21 is used as a restoring force, and the clip 2 returns to the open state while moving toward the distal end side A1.

By further pulling the proximal end portion 28 to the proximal end side A2 of the holding member 3, the engaging portion 25 is pulled in from the tightening member 32 to the proximal end side A2. Since the proximal end side A2 of the engaging portion 25 is formed to have an obtuse slope, the engaging portion 25 can be easily retracted from the tightening member 32 to the proximal end side. On the other hand, since the distal end side A1 of the engaging portion 25 is formed as an acute slope, when the engaging portion 25 is pulled from the tightening member 32 to the proximal end side A2, the engaging portion 25 and the tightening member 32 are engaged. As a result, the clip 2 cannot move to the distal end side A1, and the pair of arms 21 are locked in the closed state. Once the pair of arms 21 are locked in the closed state, the pair of arms 21 cannot return to the open state.

### [Cartridge 5]

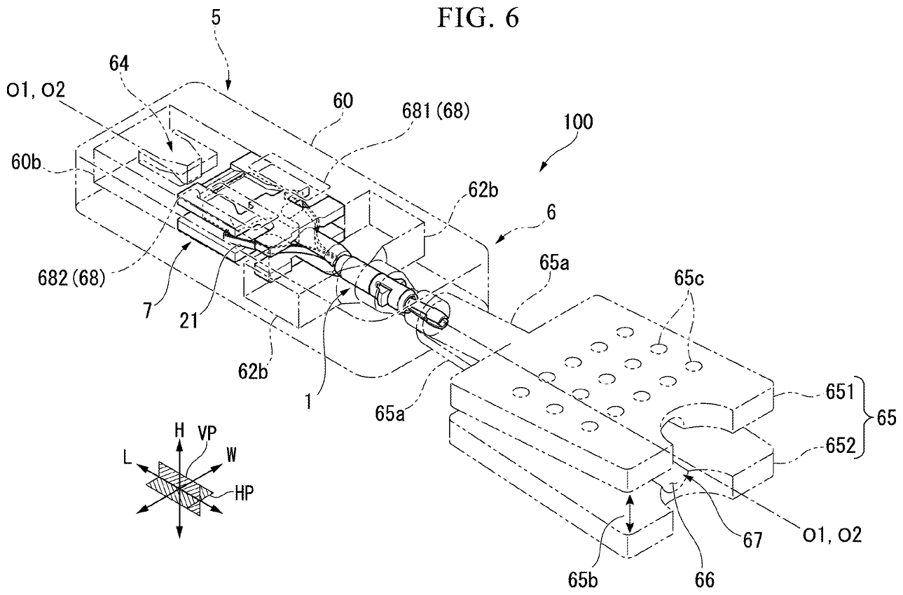

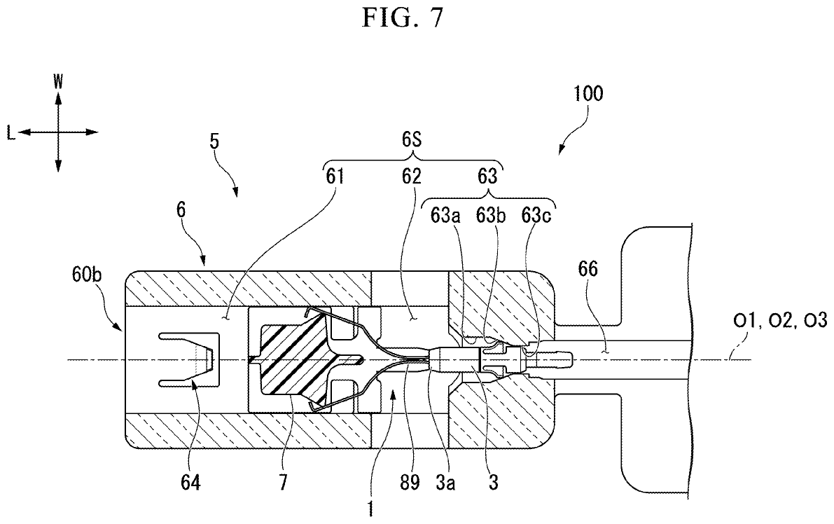

FIG. 6 is a perspective view of the cartridge 5 storing the clip unit 1. FIG. 7 is a sectional view of the cartridge 5 housing the clip unit 1. The cartridge 5 includes a case 6 and a restricting member 7. The cartridge 5 includes a length of about 50 mm in the longitudinal direction L, a width of about 10 mm to 20 mm, and a thickness (height) of about 5 mm, and is formed in a size that is easy to hold.

As shown in FIG. 6, of two directions perpendicular to the longitudinal direction L of the cartridge 5 and perpendicular to each other, one is the “width direction W” and the other is the “height direction H”. A plane horizontal to the longitudinal direction L and the width direction W is defined as a “horizontal plane HP”. A plane horizontal to the longitudinal direction L and the height direction H is defined as a “vertical plane VP”. Further, in the cartridge 5 storing the clip unit 1, the pair of arms 21 side is the distal end side of the cartridge 5, and the connecting member 4 side is the proximal end side of the cartridge 5.

The case 6 includes a case main body 60, a compression portion 65 and a sheath-connecting portion 66. The case 6 is manufactured by injection-molding a transparent resin material having moderate hardness such as ABS, PC, PP, PS, acryl, cycloolefin polymer, or the like. The case 6 is formed using a transparent resin material, and the user can easily determine whether or not the clip unit 1 exists inside.

The case main body 60 is formed in a rectangular box shape. The length in the width direction W of the case main body 60 is longer than the length in the height direction H of the case main body 60.

The case main body 60 is formed with a storage area 6S in which the clip unit 1 is stored movably in the longitudinal direction L. The storage area 6S includes a first area 61, a second area 62, and a folded portion 63. As shown in FIG. 7, the first area 61, the second area 62, and the folded portion 63 are arranged in the longitudinal direction L from the distal end to the proximal end. The first area 61, the second area 62 and the folded portion 63 are internal spaces formed symmetrically with respect to a vertical plane VP including the central axis O2 in the longitudinal direction L of the cartridge 5.

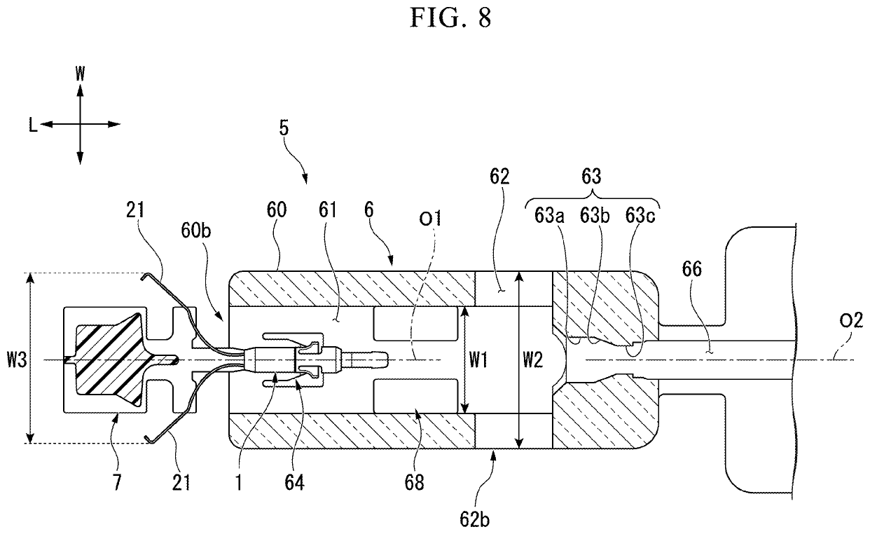

FIG. 8 is a cross-sectional view of the cartridge 5 before the clip unit 1 is stored.

The clip unit 1 is stored in the storage area 6S from the distal end opening 60b formed on the distal end side of the storage area 6S of the case main body 60. The clip unit 1 is stored in the cartridge 5 from the side of the connecting member 4.

The clip unit 1 is stored in the storage area 6S with the central axis O1 of the clip unit 1 along the longitudinal direction L, as shown in FIG. 8. The clip unit 1 is stored in the storage area 6S with the opening/closing direction P of the pair of arms 21 aligned with the width direction W of the case 6.

The first area 61 is an internal space in which the clip unit 1 is stored movably in the longitudinal direction L. The first area 61 communicates with the second area 62. The first area 61 includes an engaging portion 64 and an opening 68.

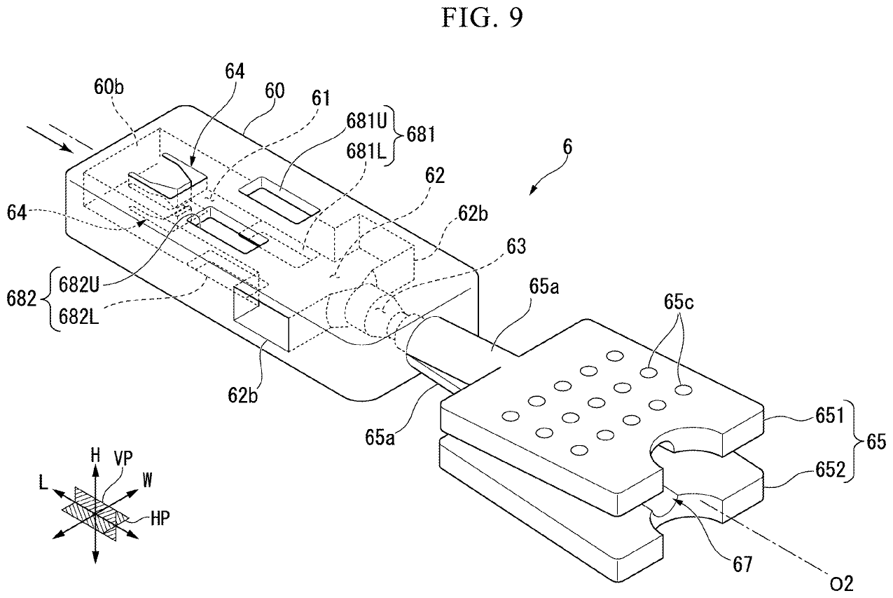

FIG. 9 is a perspective view of the case 6 in which the clip unit 1 is not stored.

The engaging portions 64 are formed on the distal end side of the first area 61 on one side (upper side) and the other side (lower side) in the height direction H. The two engaging portions 64 are formed symmetrically with respect to the horizontal plane HP including the central axis O2. The engaging portion 64 may be formed only on one side (upper side) or the other side (lower side) of the first area 61 in the case main body 60.

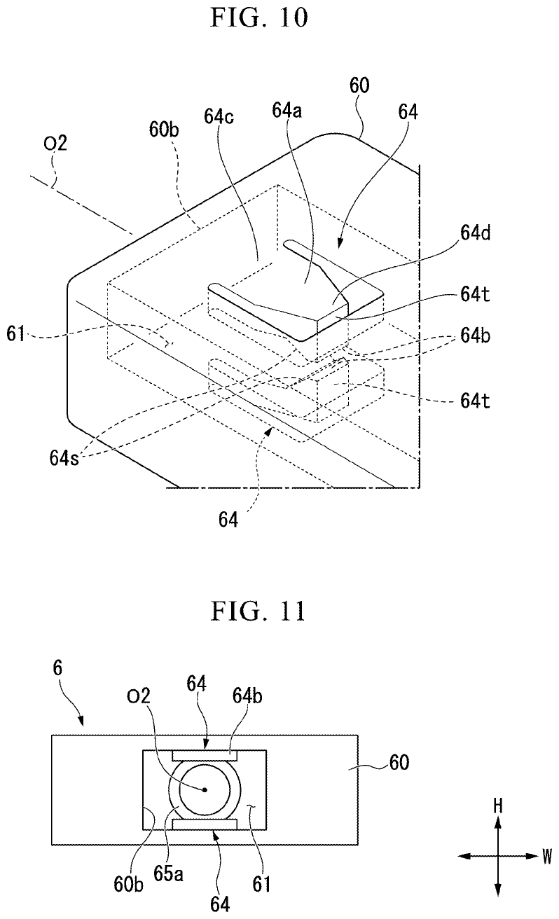

FIG. 10 is a perspective view showing the engaging portion 64.

The engaging portion 64 includes an elastic deformation portion 64a and an engaging convex portion 64b. The elastic deformation portion 64a is formed in a flat plate shape, and only an end portion 64c on the distal end side in the longitudinal direction L is fixed to the case main body 60. The elastic deformation portion 64a elastically deforms as a cantilever with an end portion 64c fixed to the case main body 60 as a fixed end.

FIG. 11 is a front view of the case 6 seen from the direction of the arrow shown in FIG. 9.

The engaging convex portion 64b is provided at an end portion 64d on the proximal end side in the longitudinal direction L of the elastic deformation portion 64a. As shown in FIG. 11, the engaging convex portion 64b protrudes toward the first area 61 of the storage area 6S when no external force acts (initial state). The engaging convex portion 64b includes an inclined surface 64s and an engaging surface 64t.

The inclined surface 64s is formed on the distal end side in the longitudinal direction L of the engaging convex portion 64b. The inclined surface 64s is inclined with respect to the central axis O2, and the normal line of the inclined surface 64s faces the distal end side of the longitudinal direction L.

The engaging surface 64t is formed on the proximal end side in the longitudinal direction L of the engaging convex portion 64b. The engaging surface 64t is formed in the plane substantially perpendicular with respect to the central axis O2, and the normal line of the inclined surface 64s faces the proximal end side of the longitudinal direction L.

The opening 68 is an opening penetrating in the height direction H provided in the case main body 60 and communicating with the first area 61. The opening 68 is provided closer to the proximal side than the engaging portion 64. The opening 68 includes a pair of first openings 681 and a pair of second openings 682.

As shown in FIG. 9, the pair of first openings 681 includes an upper first opening 681U provided on one side (upper side) in the height direction H, and a lower first opening 681L provided on the other side (lower side) in the height direction H. The upper first opening 681U and the lower first opening 681L are formed symmetrically with respect to the horizontal plane HP including the central axis O2. The upper first opening 681U and the lower first opening 681L should at least partially overlap when viewed from the height direction H.

The pair of second openings 682 includes an upper second opening 682U provided on one side (upper side) in the height direction H, and a lower second opening 682L provided on the other side (lower side) in the height direction H. The upper second opening 682U and the lower second opening 682L are formed symmetrically with respect to the horizontal plane HP including the central axis O2. The upper second opening 682U and the lower second opening 682L may at least partially overlap when viewed from the height direction H.

The pair of first openings 681 and the pair of second openings 682 are formed symmetrically with respect to the vertical plane VP including the central axis O2.

The second area 62 is an internal space in which the clip unit 1 is stored movably in the longitudinal direction L. The length in the longitudinal direction L of the second area 62 is shorter than the length in the longitudinal direction L of the first area 61. The second area 62 communicates with the folded portion 63.

As shown in FIG. 8, the length W1 in the width direction W of the first area 61 is smaller than the opening width W3 of the pair of arms 21 in the open state. Also, the length W2 in the width direction W of the second area 62 is greater than the opening width W3 of the pair of arms 21 in the open state.

The second area 62 includes openings 62b on both sides in the width direction W. The edge of the opening 62b is formed in a rectangular shape and extends in the height direction H and the longitudinal direction L.

The folded portion 63 includes an enlarged diameter portion 63a, a tapered portion 63b, and a reduced diameter portion 63c. The enlarged diameter portion 63a, the tapered portion 63b, and the reduced diameter portion 63c are arranged from the distal end to the proximal end.

The enlarged diameter portion 63a is a region that allows the elastic arm portion 44 of the connecting member 4 to elastically expand (open and close). In the enlarged diameter portion 63a, when the arrowhead hook portion 231 of the clip introduction device 200 and the connecting member 4 of the clip unit 1 are engaged with each other, the elastic arm portion 44 of the connecting member 4 can be opened and closed in a direction perpendicular to the central axis O1.

The tapered portion 63b is provided on the proximal end side of the enlarged diameter portion 63a and is formed in a tapered shape. The tapered portion 63b is expanded in diameter from the proximal side toward the distal side. Therefore, when the holding member 3 is slid from the distal end side to the proximal end side, the protruding/retracting wing 31 of the holding member 3 is accommodated inside the holding tube main body 30.

The diameter-reduced portion 63c is a region that holds the protruding/retracting wing 31 in a retracted state. The reduced diameter portion 63c is a region that does not allow the elastic arm portion 44 of the connecting member 4 to elastically expand (open and close).

When the holding member 3 is slid on the tapered portion 63b from the distal end side toward the proximal end side, the protruding/retracting wing 31 of the holding member 3 is accommodated inside the holding member 3. Therefore, the diameter-reduced portion 63c smoothly connected to the tapered portion 63b can be held in a retracted state (recessed state) of the protruding/retracting wing 31 of the holding member 3.

The compression portion 65 is a plate-like member provided at the proximal end of the case main body 60, as shown in FIG. 6. The compression portion 65 includes a first compression portion 651 and a second compression portion 652. The first compression portion 651 and the second compression portion 652 are provided facing each other in the height direction H of the case 6.

The compression portion 65 includes a connecting portion 65a that connects with the case main body 60. The connecting portion 65a separately connects the first compression portion 651 and the second compression portion 652 to the case main body 60. The connecting portion 65a is bent such that the first compression portion 651 and the second compression portion 652 are separated from each other. Thereby, a space 65b is formed between the first compression portion 651 and the second compression portion 652. The first compression portion 651 and the second compression portion 652 are spaced further apart on the proximal side than on the distal side.

The first compression portion 651 and the second compression portion 652 are, for example, about 20 mm square, and are formed in a size suitable for grasping with fingers. On the outer surfaces of the first compression portion 651 and the second compression portion 652, for example, a plurality of hemispherical concave portions 65c are formed to prevent slippage during pressing.

The sheath-connecting portion 66 is an insertion groove into which the sheath 220 can be inserted. The sheath-connecting portion 66 is an arc-shaped groove formed on the inner surfaces of the first compression portion 651 and the second compression portion 652, and communicates with the reduced diameter portion 63c of the folded portion 63 in the storage area 6S.

The user can fix the sheath 220 to the case 6 by compressing the first compression portion 651 and the second compression portion 652 while inserting the sheath 220 from a proximal end opening 67 into the sheath-connecting portion 66.

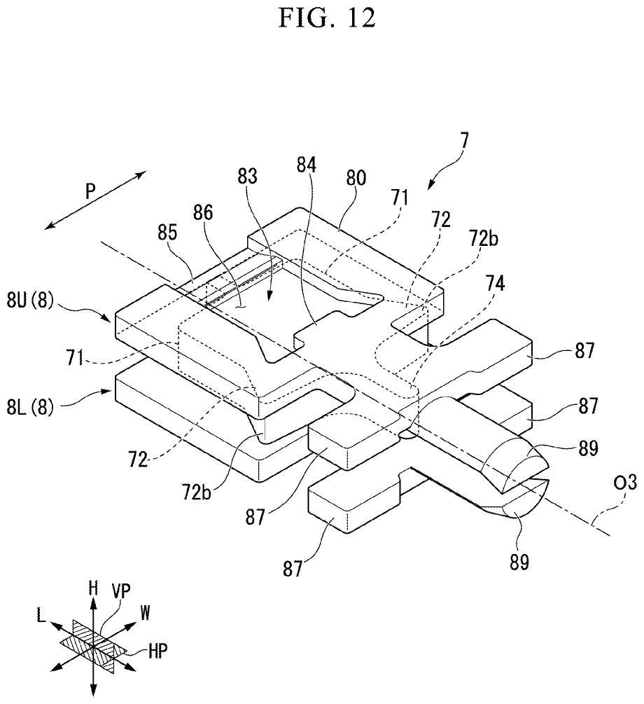

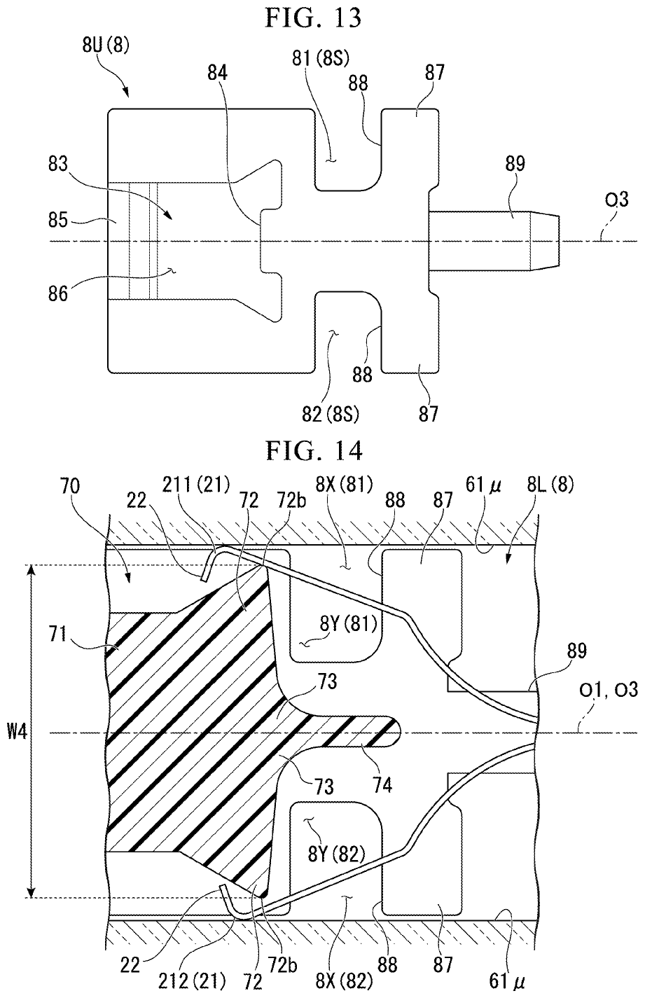

FIG. 12 is a perspective view of the restricting member 7. FIG. 13 is a plan view of the restricting member 7.

The restricting member (slider) 7 is movably stored in the first area 61 and the second area 62 together with the clip unit 1. The restricting member 7 is made of the same resin as the case 6, for example. The restricting member 7 does not have to be made of a transparent resin like the case 6. As shown in FIG. 12, the restricting member 7 is formed in a shape symmetrical with respect to a vertical plane VP including a central axis O3 in the longitudinal direction L of the restricting member 7 and a horizontal plane HP.

The restricting member 7 includes an intermediate layer member 70 and a guide member 8. The intermediate layer member 70 includes a distal end portion 71, a protruding portion 72, a tapered portion 73, and a proximal end portion 74. The distal end portion 71, the protruding portion 72, the tapered portion 73, and the proximal end portion 74 are arranged from the distal end to the proximal end along the direction of the central axis O3 of the restricting member 7.

FIG. 14 is a diagram showing the intermediate layer member 70 of the restricting member 7 grasped by the pair of arms 21.

The intermediate layer member 70 is grasped by the clip 2 with the central axis O3 substantially aligned with the central axis O1 of the clip unit 1. The clip unit 1 is stored in the storage area 6S with the clip 2 grasping the restricting member 7, as shown in FIG. 7. At this time, it may be that the central axis O1, the central axis O2, and the central axis O3 be aligned.

The distal end portion 71 is provided so as to protrude from the protruding portion 72 toward the distal end side. The distal end of the distal end portion 71 is positioned further distal than the tissue-grasping portion 22 of the clip 2 that grasps the restricting member 7. Further, the distal end of the distal end portion 71 is formed on a plane perpendicular to the central axis O3.

The protruding portion 72 is a member protruding in a direction substantially perpendicular to the central axis O3 (hereinafter also referred to as “protruding direction P”). The protruding portions 72 are provided on both sides of the central axis O3. The protruding portion 72 is grasped by the first arm 211 and the second arm 212. The opening/closing direction P of the pair of arms 21 that grasp the protruding portion 72 substantially coincides with the protruding direction P of the protruding portion 72. The first arm 211 and the second arm 212 are formed in an asymmetrical shape with respect to the central axis O1. Therefore, as shown in FIG. 14, the first arm 211 and the second arm 212 that grasp the protruding portion 72 are arranged at asymmetrical positions with respect to the central axis O3.

In the protruding portion 72, the portion that protrudes most in the protruding direction P from the central axis O3 is the maximum protruding point 72b. The length W4 between the maximum protruding points 72b is slightly smaller than the length W1 in the width direction W of the first area 61. Therefore, the restricting member 7 is maintained in a state of being held by the pair of arms 21 in the first area 61.

The tapered portion 73 is a member formed in a tapered shape. The tapered portions 73 are provided on both sides of the central axis O3. The length of the tapered portion 73 in the protruding direction P is shorter than that of the protruding portion 72. The length of the tapered portion 73 in the protruding direction P becomes shorter from the distal end side to the proximal end side.

The proximal end portion 74 is a member that protrudes from the tapered portion 73 toward the proximal end side.

The guide member 8 adjusts the position of the restricting member 7 in the height direction H so that the protruding portion 72 is grasped by the pair of arms 21. Further, the guide member 8 guides the restricting member 7 so that the restricting member 7 moves along the longitudinal direction L in the first area 61. The guide member 8 includes an upper layer guide member 8U provided on one side (upper side) in the height direction H and a lower layer guide member 8L provided on the other side (lower side) in the height direction H. The intermediate layer member 70 is sandwiched in the height direction H between the upper layer guide member 8U and the lower layer guide member 8L. The upper layer guide member 8U and the lower layer guide member 8L are formed in a shape symmetrical with respect to the horizontal plane HP containing the central axis O3.

The upper guide member 8U includes an engaging portion 80, a wing portion 87, and a holding portion 89. The engaging portion 80, the wing portion 87, and the holding portion 89 are arranged along the central axis O3 direction of the restricting member 7 from the distal end to the proximal end.

An engaging concave portion 83 is formed on one side (upper side) in the height direction H of the engaging portion 80 of the upper guide member 8U. The engaging concave portion is a recess that engages with the engaging convex portion 64b of the engaging portion 64.

The engaging concave portion 83 is formed in a substantially rectangular shape when viewed from above in the height direction H. A region of the engaging portion 80 where the engaging concave portion 83 is not formed is formed in a U-shape in plan view.

As shown in FIG. 12, the engaging concave portion 83 includes an engaging surface 84 on the proximal end side in the longitudinal direction L that engages with the engaging convex portion 64b of the engaging portion 64. The engaging surface 84 is formed on a plane substantially perpendicular to the central axis O3. On the other hand, the engaging concave portion 83 includes a notch portion 85 formed on the distal end side in the longitudinal direction L. Therefore, a recessed space 86 of the engaging concave portion 83 communicates with the space on the distal end side in the longitudinal direction L.

When the restricting member 7 moves from the proximal end side to the distal end side of the first area 61, the engaging surface 64t of the engaging portion 64 passes through the notch portion 85 and the recessed space 86 and hits the engaging surface 84.

The wing portions 87 are a pair of wing-shaped members that are provided on the proximal end side of the engaging portion 80 and extend on both sides in the width direction W. As shown in FIG. 13, the length in the width direction W of the wing portions 87 extending on both sides in the width direction W is substantially equal to the length in the width direction W of the engaging portion 80. A notch portion 88 that is notched toward the inside in the width direction W is formed between the wing portion 87 and the engaging portion 80. The notch portion 88 forms a through-space 8S that penetrates the upper layer guide member 8U in the height direction H.

As shown in FIG. 14, in plan view in the height direction H, at least a portion of the pair of arms 21 grasping the protruding portion 72 of the intermediate layer member 70 overlaps from the through-space 8S formed by the notch portion 88. In plan view viewed from the height direction H, the through-space 8S is classified into an outer through-space 8X outside the pair of arms 21 in the width direction W and an inner through-space 8Y inside the pair of arms 21 in the width direction W.

The through-space 8S that overlaps at least a portion of the first arm 211 that grasps the protruding portion 72 of the intermediate layer member 70 in plan view from the height direction H is also referred to as a first through-space 81. The through-space 8S that overlaps at least a portion of the second arm 212 that grasps the protruding portion 72 of the intermediate layer member 70 in plan view in the height direction H is also referred to as a second through-space 82.

The holding portion 89 is a member that restricts the minimum approach distance between the clip 2 and the holding member 3. The holding portion 89 is provided on the proximal end side of the wing portion 87. As shown in FIG. 7, the holding portion 89 is engaged with the edge of the distal end opening 3a of the holding member 3, so that it cannot enter the internal space of the holding member 3 through the distal end opening 3a. Therefore, even when the clip 2 is pulled closer to the holding member 3, the holding portion 89 engages with the edge of the distal end opening 3a to restrict the minimum approach distance between the clip 2 and the holding member 3.

The lower layer guide member 8L includes an engaging portion 80, a wing portion 87, and a holding portion 89, similar to the upper layer guide member 8U. The lower layer guide member 8L is formed in a shape symmetrical with the upper layer guide member 8U with respect to the horizontal plane HP including the central axis O3.

The restricting member 7 is stored in the first area 61 while being held by the pair of arms 21. As shown in FIG. 8, the length W1 in the width direction W of the first area 61 is smaller than the opening width W3 of the pair of arms 21 in the open state. Therefore, the pair of arms 21 grasp the restricting member 7 from the open state to the closed state. The opening width of the pair of arms 21 is approximately the length W1 in the width direction W of the first area 61. The pair of arms 21 abut on the case main body 60 in the opening/closing direction P.

### [Method of Loading Clip Unit 1]

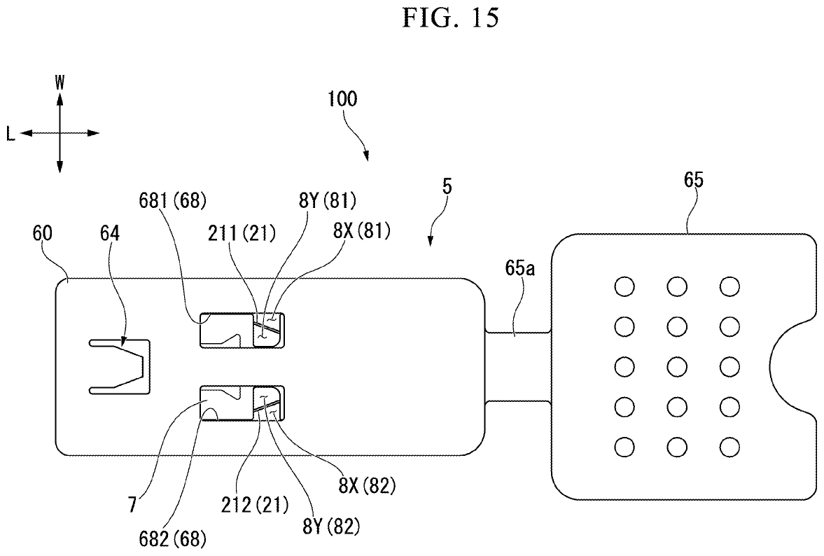

Next, the action of the cartridge system 100 will be described. FIGS. 15 to 26 are diagrams showing a method of loading the clip unit 1 into the clip introduction device 200 using the cartridge 5.

FIG. 15 is a diagram showing the cartridge 5 in which the clip unit 1 is stored.

In plan view in the height direction H, the openings 68 (the pair of first openings 681 and the pair of second openings 682) are formed at a position where at least part of the pair of arms 21 of the clip unit 1 positioned at a predetermined position is exposed. In the present embodiment, in plan view from the height direction H, the opening 68 is formed at a position overlapping the through-space 8S (the first through-space 81, the second through-space 82) of the restricting member 7 grasped by the clip unit 1 positioned at a predetermined position. Specifically, the pair of first openings 681 are formed at positions overlapping the first through-spaces 81 of the restricting member 7 grasped by the clip unit 1 positioned at a predetermined position. The pair of second openings 682 are formed at positions overlapping the second through-spaces 82 of the restricting member 7 grasped by the clip unit 1 positioned at a predetermined position.

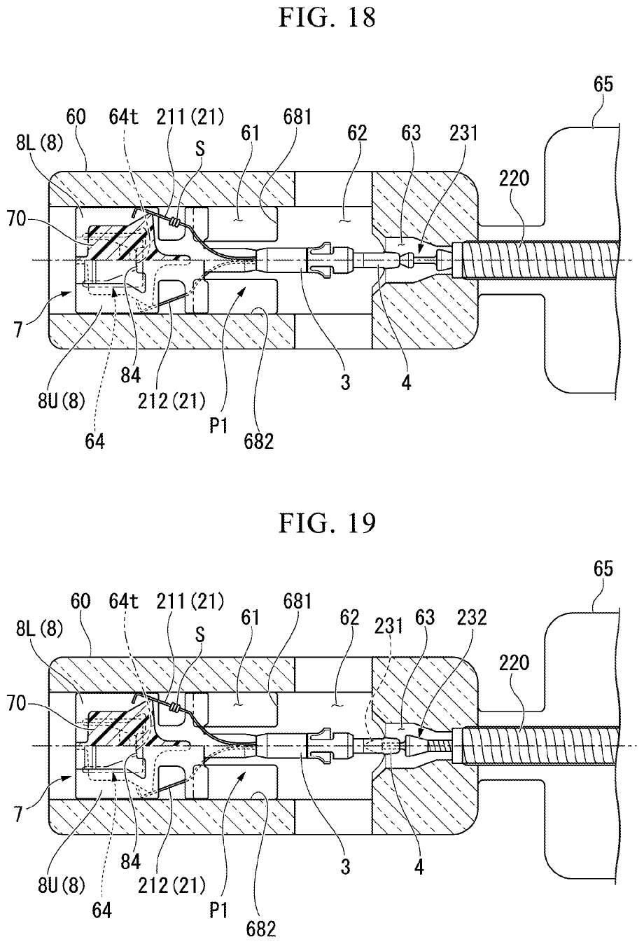

For example, as shown in FIG. 18, the predetermined position is the distal end side position P1 (the most proximal position).

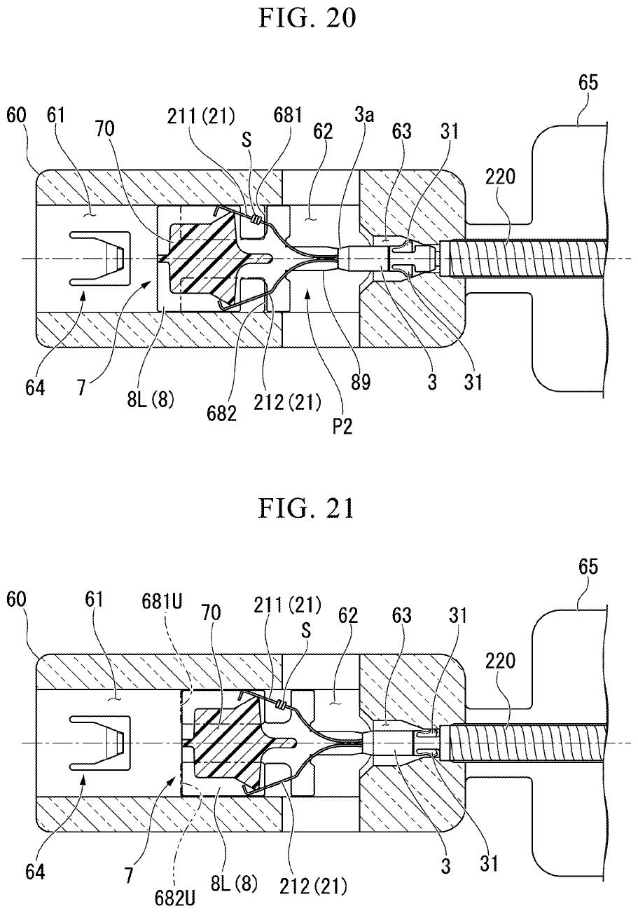

The predetermined position is, for example, a proximal side position P2 (most proximal side position) where the clip unit 1 engages with the case 6 on the proximal side in the longitudinal direction A in the storage area 6S, as shown in FIG. 20. Specifically, the proximal position P2 is the position where the protruding/retracting wing 31 engages with the folded portion 63 of the case 6.

In plan view in the height direction H, the openings 68 (the pair of first openings 681 and the pair of second openings 682) are desirably formed at a position where at least a part of the pair of arms 21 of the clip unit 1 arranged at any position is exposed. In the present embodiment, in plan view viewed from the height direction H, the opening 68 is desirably formed at a position overlapping with the through-space 8S (the first through-space 81, the second through-space 82) of the restricting member 7 grasped by the clip unit 1 arranged at any position. In this case, at least a portion of the pair of arms 21 is exposed through the opening 68 regardless of the position of the clip unit 1 stored in the cartridge 5.

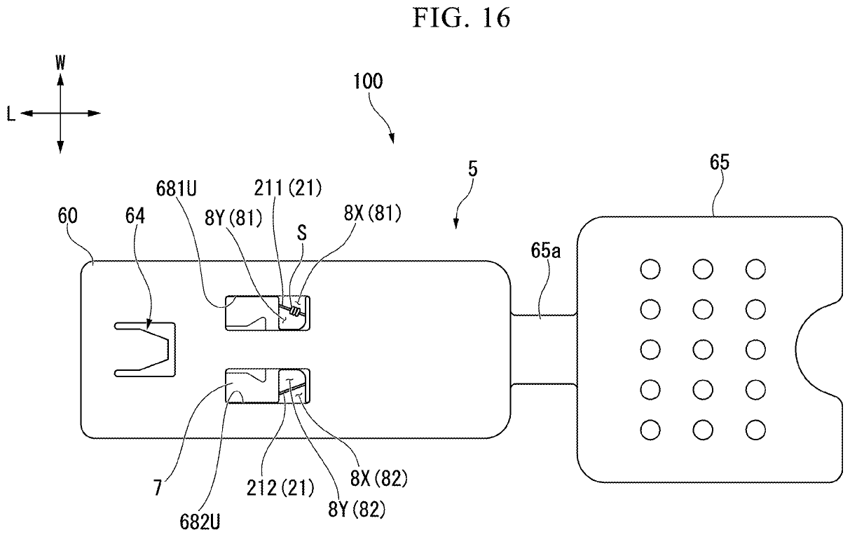

FIG. 16 is a diagram showing the appearance of the cartridge 5.

The user exposes at least part of the pair of arms 21 through the opening 68. At this time, the user may attach the thread S to the pair of arms 21 using the opening. In that case, the user inserts the thread S from the upper first opening 681U, passes through the inner through-space 8Y of the first through-space 81, and takes out the thread S from the lower first opening 681L. Next, the user inserts the thread S from the lower first opening 681L, passes it through the outer through-space 8X of the first through-space 81, and takes out the thread S from the upper first opening 681U. The user repeats this to wind the thread S around the first arm 211 and tie the thread S on the side of the upper first opening 681U.

Alternatively, the user inserts the thread S from the upper first opening 681U, passes it through the outer through-space 8X of the first through-space 81, and takes out the thread S from the lower first opening 681L. Next, the user inserts the thread S from the lower first opening 681L, passes it through the inner through-space 8Y of the first through-space 81, and takes out the thread S from the upper first opening 681U. The user repeats this to wind the thread S around the first arm 211 and tie the thread S on the side of the upper first opening 681U.

An inner through-space 8Y and an outer through-space 8X are formed in the first through-space 81, by which the user can easily tie the thread S to the first arm 211.

The user may pass the thread S through the pair of second openings 682 instead of the pair of first openings 681 and tie the thread S to the second arm 212.

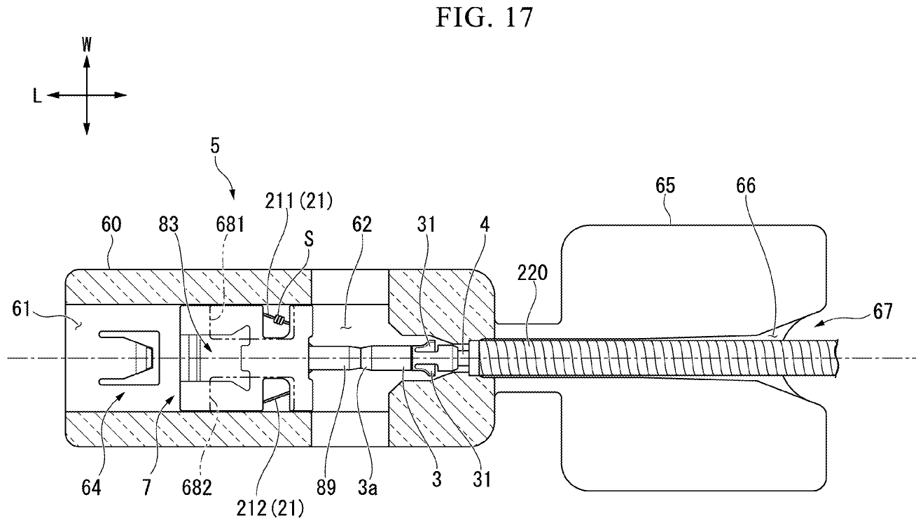

As shown in FIG. 17, the user inserts the sheath 220 of the clip introduction device 200 into the sheath-connecting portion 66 of the case 6 through the proximal end opening 67. The user compresses the sheath 220 with the compression portion 65 to fix the sheath 220 to the case 6.

As shown in FIG. 18, the user operates the operation portion 240 to advance the operation wire 230 with respect to the sheath 220, thereby advancing the arrowhead hook portion 231. As the arrowhead hook portion 231 advances the connecting member 4, the restricting member 7 and the clip unit 1 advance. As the restricting member 7 moves from the proximal end side to the distal end side, the engaging surface 84 of the restricting member 7 hits and engages with the engaging surface 64t of the engaging portion 64. As a result, the clip unit 1 cannot move forward beyond that position. That is, the engaging portion 64 restricts passage of the clip unit 1 moving toward the distal end. As shown in FIG. 19, the advancing arrowhead hook portion 231 is sandwiched between the elastic arm portions 44 of the connecting member 4 of the clip unit 1 and connected to the connecting member 4.

The engaging surface 84 of the restricting member 7 is formed on a plane substantially perpendicular to the central axis O3. The engaging surface 64t of the engaging portion 64 is formed on a plane substantially perpendicular to the central axis O2. Therefore, the restricting member 7 moving from the proximal side to the distal side is less likely to push the engaging portion 64 to one side (upper side) in the height direction H. Further, even if the engaging surface 84 of the restricting member 7 abuts against and engages with the engaging surface 64t of the engaging portion 64, the central axis O1 of the clip unit 1 is less likely to deviate from the central axis O2 of the storage area 6S.

Even if the restricting member 7 does not include the engaging concave portion 83, the restricting member 7 moves from the proximal end side to the distal end side to collide with and engage with the engaging surface 64t of the engaging portion 64. As a result, the advancing arrowhead hook portion 231 is connected to the connecting member 4 in the same manner as described above.

As shown in FIG. 20, when the user pulls the operation wire 230, the clip 2 of the clip unit 1 is pulled to the proximal end side by the connecting member 4 connected to the arrowhead hook portion 231. The hook 41f of the connecting member 4 pulls the proximal end portion 28 of the clip 2 without breaking. The restricting member 7 moves in the first area 61 of the storage area 6S while being in contact with the pair of arms 21. Since the holding portion 89 of the restricting member 7 engages with the edge of the distal end opening 3a of the holding member 3, the holding member 3 is pulled to the proximal end side together with the clip 2.

The pair of arms 21 abut on the case main body 60 in the opening/closing direction P in the first area 61. By the frictional force generated by the contact between the pair of arms 21 and the case main body 60, the clip 2 is drawn into the internal space of the holding member 3, and it is possible to reliably prevent the clip 2 from being locked in the closed state by the holding member 3.

When the clip 2 is pulled to the proximal end side, the holding portion 89 of the restricting member 7 engages with the edge of the distal end opening 3a of the holding member 3, so the minimum approach distance between the clip 2 and the holding member 3 is restricted. The holding portion 89 abuts on the holding member 3 while being in contact with the pair of arms 21 by being pulled by the operation wire 230, and restricts the relative movement of the restricting member 7 with respect to the holding member 3. This also reliably prevents the clip 2 pulled to the proximal end side by the connecting member 4 from being drawn into the internal space of the holding member 3 and locked in the closed state by the holding member 3.

The user pulls the clip unit 1 further to the proximal side. As shown in FIG. 21, the holding member 3 passes through the folded portion 63. The holding member 3 is slid along the diameter-reduced portion 63c from the distal end side toward the proximal end side, and the protruding/retracting wing 31 of the holding member 3 is housed inside the holding tube main body 30. The holding member 3 with the protruding/retracting wings 31 housed inside the holding tube main body 30 is pulled into the sheath 220.

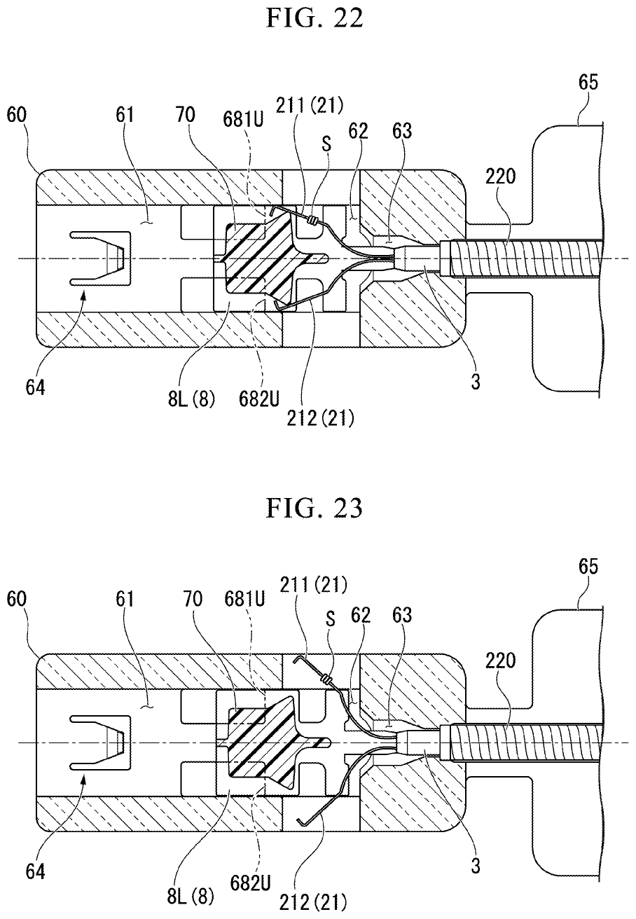

As shown in FIG. 22, when the protruding/retracting wings 31 of the holding member 3 are housed inside the holding member 3, the contact points between the pair of arms 21 and the case main body 60 are located in the first area 61. That is, the pair of arms 21 grasp the restricting member 7 and are not drawn into the internal space of the holding member 3 until the protruding/retracting wing 31 is housed inside the holding member 3.

As shown in FIG. 23, the user further pulls the operation wire 230 to pull the restricting member 7 to the second area 62. The length W2 in the width direction W of the second area 62 is greater than the opening width W3 of the pair of arms 21 in the open state. Therefore, the restricting member 7 is not grasped by the pair of arms 21 in the second area 62.

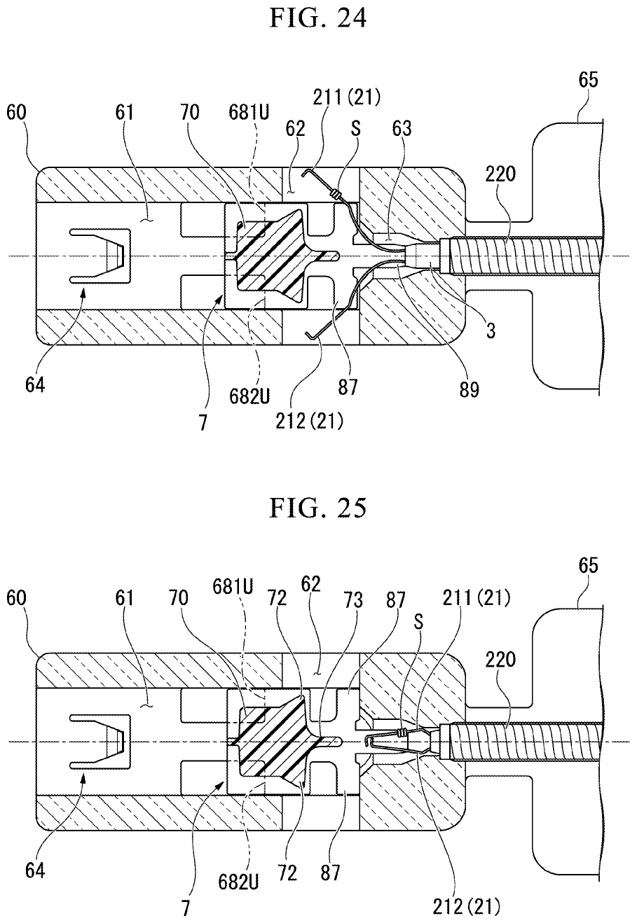

As shown in FIG. 24, the user pulls the operation wire 230 further. The wing portion 87 of the restricting member 7 engages with the end face of the second area 62 on the proximal side. As a result, the restricting member 7 is not pulled further to the proximal end side.

As shown in FIG. 25, the user pulls the operation wire 230 further. The clip 2 is separated from the restricting member 7 and pulled toward the proximal end. The restricting member 7 includes a tapered portion 73 formed on the proximal end side of the protruding portion 72 grasped by the pair of arms 21. Therefore, the pair of arms 21 are less likely to get caught on the restricting member 7 when the restricting member 7 is pulled toward the proximal end side.

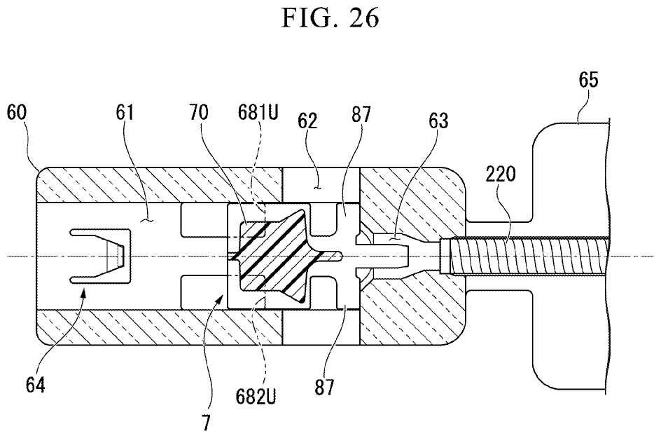

As shown in FIG. 26, the user pulls the operation wire 230 further. Since the pair of arms 21 do not grasp the restricting member 7, the minimum approach distance between the clip 2 and the holding member 3 is not restricted. The clip 2 pulled to the proximal end side is pulled into the sheath 220 while being pulled into the internal space of the holding member 3. The clip 2 may be pulled into sheath 220 without being pulled into the interior space of holding member 3. When the user uses the opening to attach the thread S to the pair of arms 21, this operation completes the loading of the clip unit 1 with the thread S tied to the clip 2 into the clip introduction device 200. The user releases the squeezing of the sheath 220 by the compression portion 65 and pulls out the sheath 220 from the case 6. By pulling out the sheath 220 from the case 6, the thread S is pulled out through the proximal end opening 67 of the case 6.

### [Method of Storing Clip Unit 1]

Next, a method for storing the clip unit 1 in the cartridge 5 will be described. FIGS. 27 to 30 are diagrams showing a method of storing the clip unit 1 in the cartridge 5.

As shown in FIG. 27, the user closes the pair of arms 21 by hand or the like and causes the pair of arms 21 to grasp the restricting member 7. Next, the user inserts the clip unit 1 holding the restricting member 7 into the storage area 6S of the cartridge 5 through the distal end opening 60b.

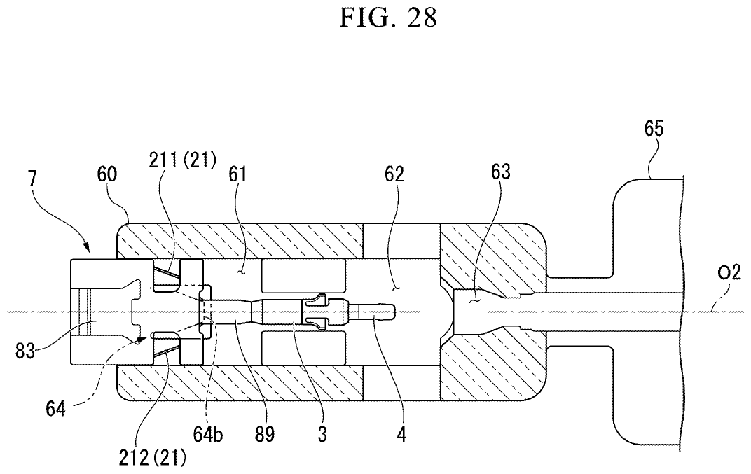

As shown in FIG. 28, the user moves the clip unit 1 to the proximal side by pushing the restricting member 7 to the proximal side. A length W1 in the width direction W of the first area 61 is smaller than an opening width W3 of the pair of arms 21 in the open state. Therefore, when the pair of arms 21 holding the restricting member 7 are inserted into the first area 61, the first arm 211 contacts one end in the width direction W of the distal end opening 60b, and the second arm 212 contacts the other end in the width direction W of the distal end opening 60b. When the user pushes the restricting member 7 further toward the proximal end side, the pair of arms 21 in contact with the distal end opening 60b are further closed and inserted into the first area 61.

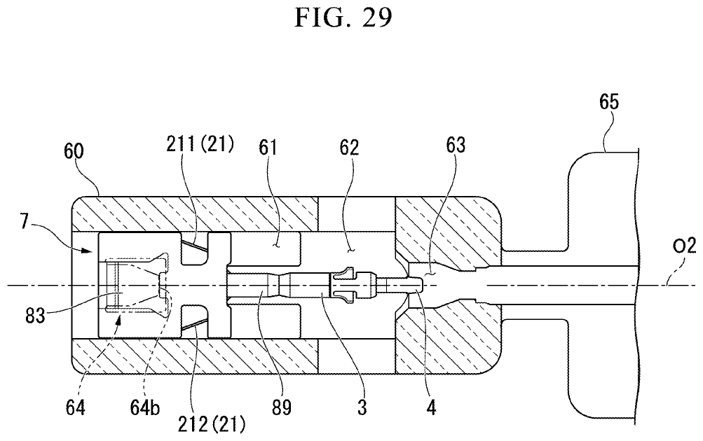

As shown in FIG. 29, when the restricting member 7 moves near the engaging portion 64 of the case 6 from the distal end side toward the proximal end side, the restricting member 7 pushes the engaging portion 64 to one side (upper side) in the height direction H so as to pass through. As shown in FIG. 10, the engaging portion 64 is elastically deformed as a cantilever with an end portion 64c on the distal end side in the longitudinal direction L as a fixed end and an end portion 64d on the proximal end side in the longitudinal direction L as a free end. Therefore, the restricting member 7 moving from the distal end side to the proximal end side is likely to elastically deform the engaging portion 64 and push it away.

As shown in FIG. 10, an inclined surface 64s is formed on the distal end side in the longitudinal direction L of the engaging convex portion 64b. When the restricting member 7 moving from the distal end side to the proximal end side comes into contact with the engaging convex portion 64b of the engaging portion 64, the restricting member 7 contacts the inclined surface 64s. Therefore, the restricting member 7 moving from the distal end side to the proximal end side easily pushes the engaging portion 64 to one side (upper side) in the height direction H.

Even if the restricting member 7 does not include the engaging concave portion 83, by moving from the distal end side to the proximal end side, the restricting member 7 can push aside the engaging portion 64 to one side (upper side) in the height direction H so as to pass through. However, since the restricting member 7 includes the engaging concave portion 83, the distance through which the restricting member 7 is passed while pushing the engaging portion 64 upward is short, making it easy for the user to operate.

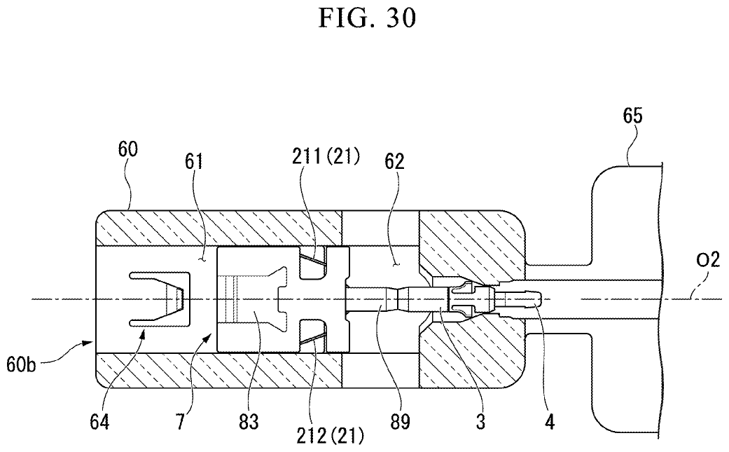

As shown in FIG. 30, after the restricting member 7 passes near the engaging portion 64 of the case 6, the engaging portion 64 returns to its initial state.

### [Shipping Mode of Cartridge 5]

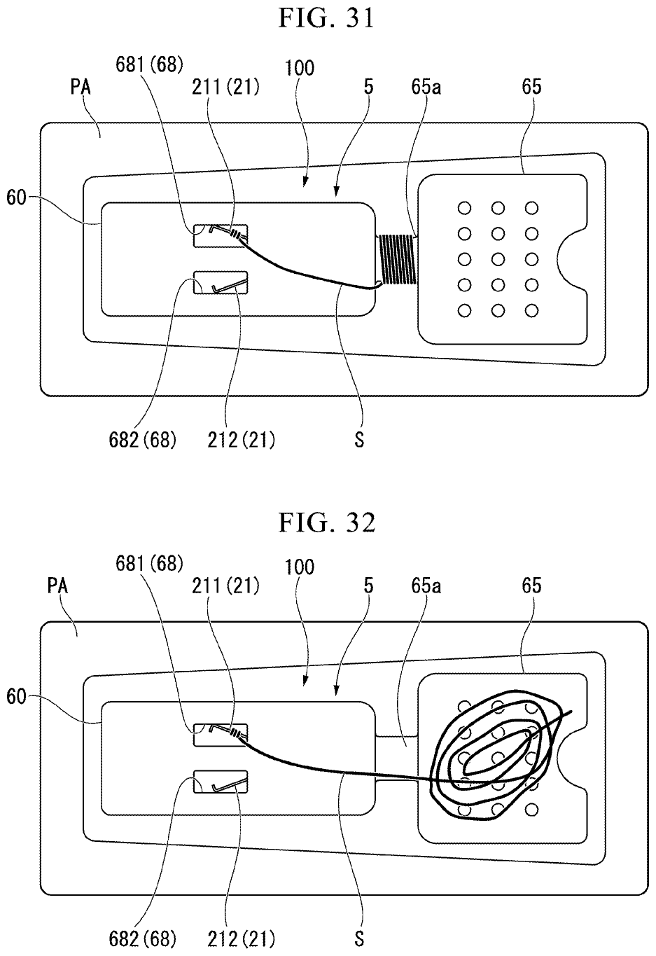

FIGS. 31 to 34 show the package PA containing the cartridge 5.

The cartridge system 100 (clip unit 1 and cartridge 5) is housed in a package PA made of translucent material and shipped. The cartridge 5 may be accommodated in the package P in a state in which the clip unit 1 to which the thread S is tied is stored.

The cartridge system 100 shown in FIG. 31 is housed in the package PA with one end of the thread S tied to the first arm 211 and the other portion of the thread S wound around the connecting portion 65a of the case 6 of the cartridge 5. Other portions of the thread S may be wound around other portions of the case 6.

The cartridge system 100 shown in FIG. 32 is housed in the package PA with one end of the thread S tied to the first arm 211 and the other portion of the thread S resting on the case 6 of the cartridge 5.

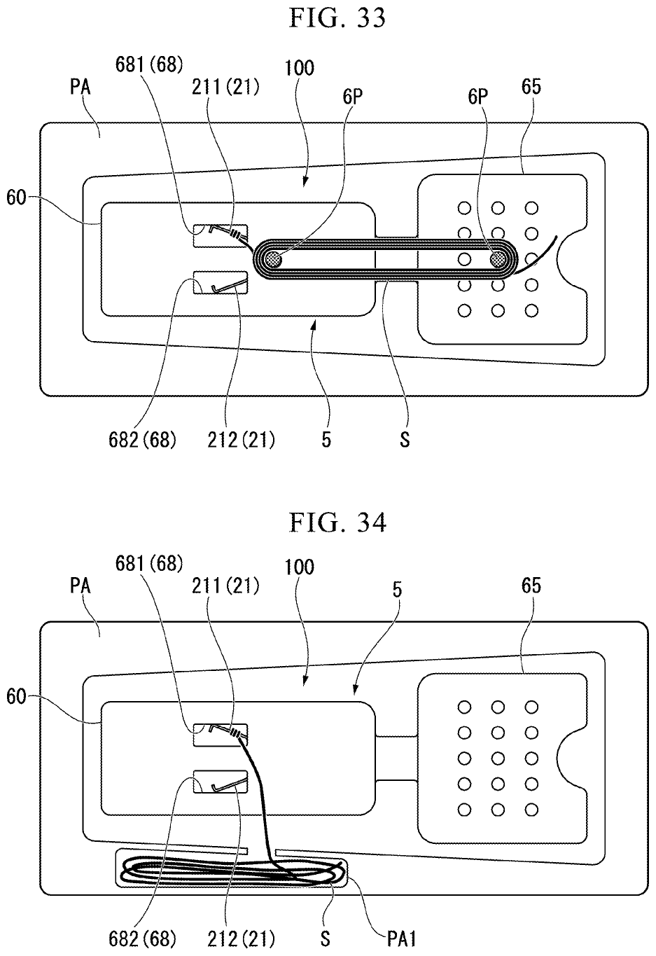

The cartridge system 100 shown in FIG. 33 is housed in the package PA with one end of the thread S tied to the first arm 211 and the other portion of the thread S wound around a protrusion 6P formed on the case 6 of the cartridge 5.

The cartridge system 100 shown in FIG. 34 is housed in a package PA with one end of the thread S tied to the first arm 211 and the other portion of the thread S stored in a sterile pack PA1 provided inside the package PA.

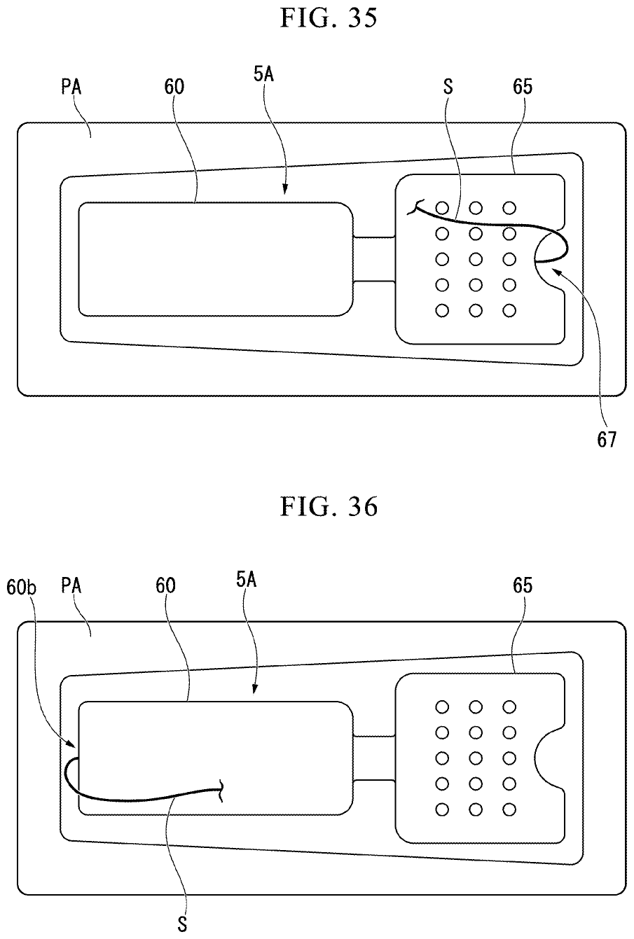

FIGS. 35 and 36 show the package PA containing the cartridge 5A without the opening 68. The cartridge 5A is the same member as the cartridge 5 except that it does not include the opening 68. Even without the opening 68, the cartridge 5A can be accommodated in the package PA with the clip unit 1 to which the string S is tied.

The cartridge 5A shown in FIG. 35 stores the clip unit 1 to which the thread S is tied, and is housed in the package PA with the thread S protruding from the proximal end opening 67.

The cartridge 5A shown in FIG. 36 stores the clip unit 1 to which the thread S is tied, and is housed in the package PA with the thread S protruding from the distal end opening 60b.

According to the cartridge system 100 of this embodiment, the clip 2 of the clip unit 1 stored in the cartridge 5 can be visually recognized, and the thread S can be easily tied to the clip 2, for example. The user can easily tie the thread S to the clip 2 of the clip unit 1 without taking out the clip unit 1 from the cartridge 5.

As described above, the first embodiment of the present disclosure has been described in detail with reference to the drawings, but the specific configuration is not limited to this embodiment, and design changes and the like are included within the scope of the present disclosure. Also, the constituent elements shown in the above-described embodiment and modifications shown below can be combined as appropriate.

### Modification 1

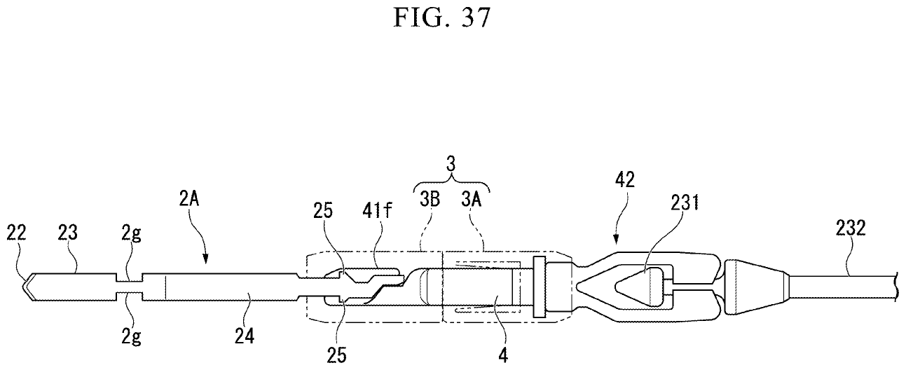

FIG. 37 is a diagram showing a clip 2A as a modification of the clip 2.

The clip 2A includes a concave portion 2g on which the tied thread S is hooked. The concave portion 2g may be positioned so as to overlap the opening 68 when viewed from the height direction. It is difficult for the thread S hooked on the concave portion 2g and tied to come off from the clip 2A. The clip 2 may include the concave portion 2g where the thread S is easily caught.

### Modification 2

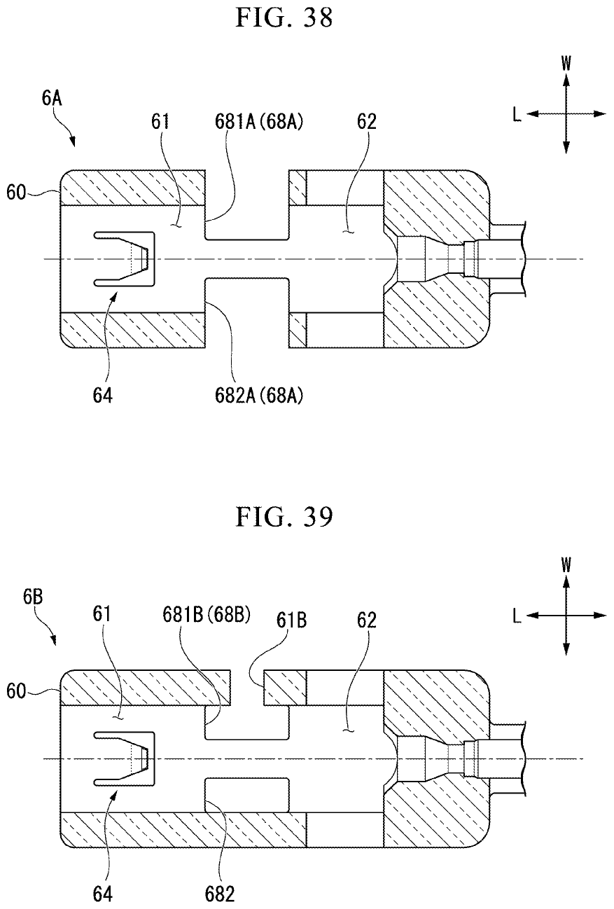

FIG. 38 is a diagram showing a case 6A, which is a modification of case 6.

The case 6A includes openings 68A (a pair of first openings 681A and a pair of second openings 682A). The openings 68A are formed in a notch shape notched toward the inside of the width direction W in the case main body 60.

### Modification 3

FIG. 39 is a diagram showing a case 6B, which is a modification of case 6.

The case 6B includes openings 68B (a pair of first openings 681B and a pair of second openings 682). The pair of first openings 681B are openings penetrating in the height direction H provided in the case main body 60 and communicating with the first area 61. The pair of first openings 681B includes a path 61B that communicates with the outside of the case 6B at a portion in the longitudinal direction L.

### Modification 4

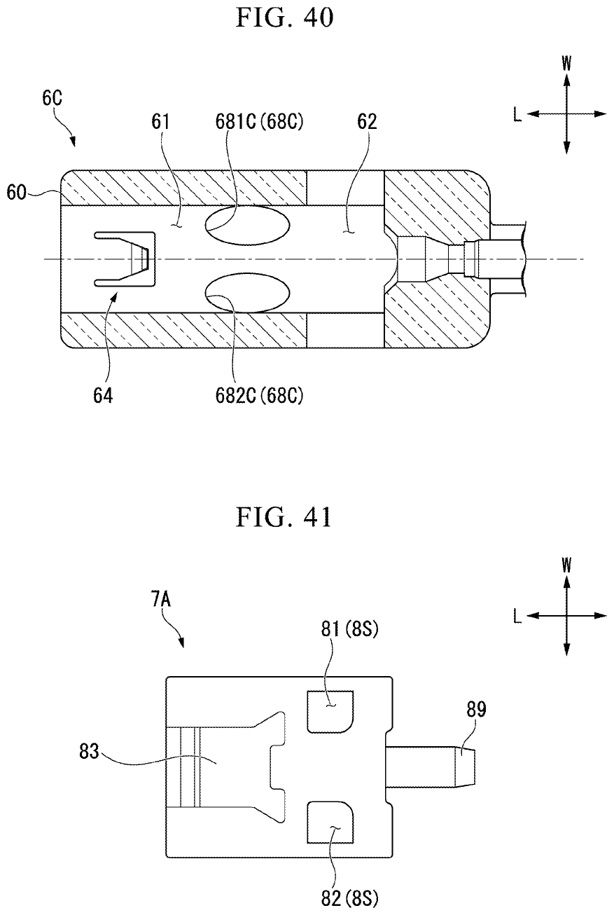

FIG. 40 is a diagram showing a case 6C, which is a modification of case 6.

The case 6C includes openings 68C (a pair of first openings 681B and a pair of second openings 682). The opening 68C is formed in an elliptical shape when viewed from above in the height direction H.

### Modification 5

FIG. 41 is a diagram showing a restricting member 7A as a modification of the restricting member 7.

The through-space 8S of the restricting member 7A is formed by a substantially quadrangular through-hole penetrating in the height direction H.

### Modification 6

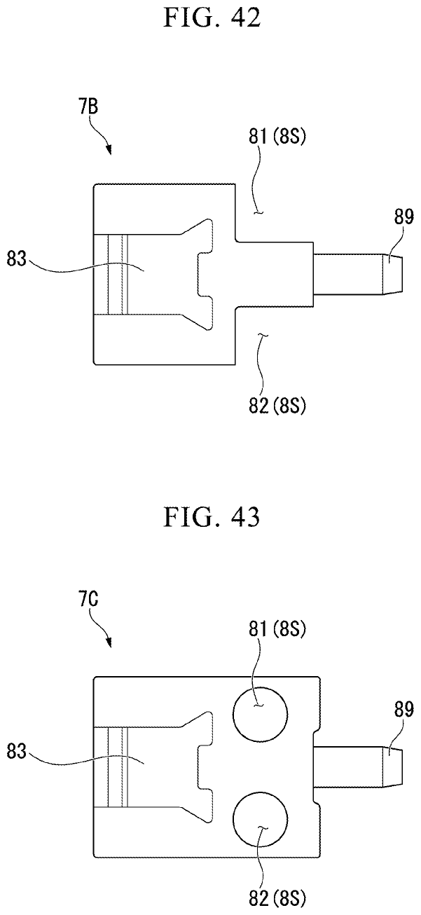

FIG. 42 is a diagram showing a restricting member 7B as a modification of the restricting member 7.

The restricting member 7B does not include the wing portion 87 as compared with the restricting member 7.

### Modification 7

FIG. 43 is a diagram showing a restricting member 7C, which is a modification of the restricting member 7.

The through-space 8S of the restricting member 7C is formed by a circular through-hole that penetrates in the height direction H.

### Second Embodiment

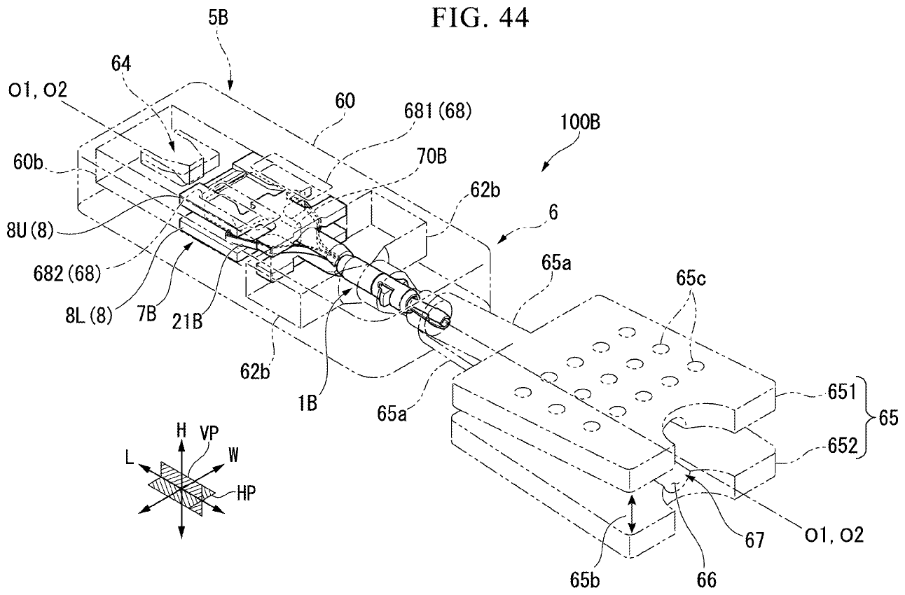

A second embodiment of the present disclosure will be described with reference to FIGS. 44 to 49. In the following description, the same reference numerals are given to the same configurations as those already described, and duplicate descriptions will be omitted. A cartridge system 100B according to the second embodiment is the same as the cartridge system 100 according to the first embodiment, except that the shapes of the pair of arms 21 and the restricting member 7 are specified in more detail.

FIG. 44 is a perspective view of a cartridge 5B housing a clip unit 1B.

The cartridge system 100B includes the clip unit 1B and the cartridge 5B that accommodates the clip unit 1B. The cartridge system 100B is a support system for easily loading the clip introduction device 200 with the clip unit 1B.

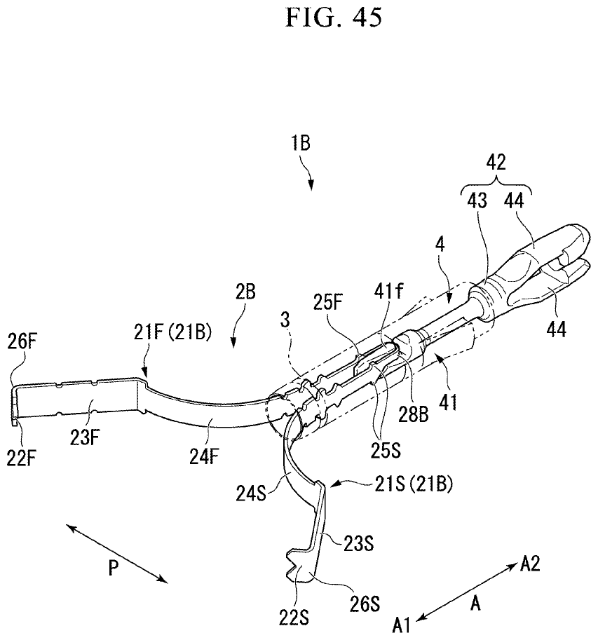

FIG. 45 is a perspective view of the clip unit 1B.

The clip unit 1B includes a clip 2B, a holding member 3, and a connecting member 4. In the following description, the side of the clip 2B in the longitudinal direction A of the clip unit 1B is referred to as the distal end side (distal side) A1 of the clip unit 1B, and the side of the connecting member 4 is referred to as the proximal end side (proximal side) A2 of the clip unit 1B.

The clip 2B is formed by bending a metal plate at the center. The clip 2B includes a pair of arms 21B that can be opened and closed, and a proximal end portion 28B that connects the pair of arms 21B. The proximal end side A2 of the clip 2B is inserted into the internal space 38 of the holding member 3.

The pair of arms 21B includes a first arm 21F and a second arm 21S shorter than the first arm 21F. The first arm 21F and the second arm 21S are arranged on both sides of the central axis O1 in the longitudinal direction A of the clip unit 1B.

The first arm 21F includes a first claw 22F, a flat plate-like first arm main body 23F, a first sliding portion 24F, and a first engaging portion 25F from the distal end side A1 toward the proximal end side A2. The first claw 22B is supported by the first arm body 23F via a first bending portion 26F that bends inward (the closing side in the opening/closing direction P). The first sliding portion 24F is a portion that is elastically deformed when the pair of arms 21B are pulled into the holding member 3.

The second arm 21S includes a second claw 22S, a flat plate-shaped second arm main body 23S, a second sliding portion 24S, and a second engaging portion 25S from the distal end side A1 toward the proximal end side A2. The second claw 22S is supported by the second arm body 23S via a second bending portion 26S that bends inward (the closing side in the opening/closing direction P). The second sliding portion 24S is a portion that is elastically deformed when the pair of arms 21B is pulled into the holding member 3.

The tissue-grasping portion 22 of the first arm 211 and the tissue-grasping portion 22 of the second arm 212 are formed in an asymmetrical shape with respect to the central axis O1. Therefore, when attaching the clip 2B to the connecting member 4, the user can easily grasp the orientation of attaching the clip 2B to the connecting member 4.

The first engaging portion 25F and the second engaging portion 25S are members that can be engaged with the tightening member 32 of the holding member 3. The distal end sides A1 of the first engaging portion 25F and the second engaging portion 25S are formed to have an obtuse slope with respect to the longitudinal direction A, and the proximal end sides A2 are formed to have an acute angle with respect to the longitudinal direction A.

The proximal end portion 28B connects the first arm 21F and the second arm 21S. The first arm 21F and the second arm 21S connected by the proximal end portion 28B are provided so as to be openable and closable toward the distal end side A1. The proximal end portion 28B is bent to have a U-shape and is connected to the hook 41f of the connecting member 4. The proximal end portion 28B is urged to open the pair of arms 21B. Therefore, the pair of arms 21B of the clip 2B have a self-expanding force in the opening/closing direction P.

The proximal end portion 28B and the hook 41f of the connecting member 4 are connected to the proximal end portion 28B formed in a U-shape in the same manner as the connection mode between the proximal end portion 28 and the hook 41f of the connecting member 4 in the first embodiment. The hook 41f of the member 4 is inserted and connected.

The cartridge 5B, as shown in FIG. 6, is used to load the stored clip unit 1B into the clip introduction device 200 in the same manner as the cartridge 5 of the first embodiment. The cartridge 5B includes a case 6 and a restricting member 7B.

The restricting member (block) 7B is movably stored in the first area 61 and the second area 62 of the storage area 6S together with the clip unit 1B, like the restricting member 7 of the first embodiment. The restricting member 7B is stored in the storage area 6S so as to be movable in the longitudinal direction L in the first area 61 and the second area 62 together with the clip unit 1B. Also, the restricting member 7B is stored in the storage area 6S in a separably engaged state with the clip unit 1B.

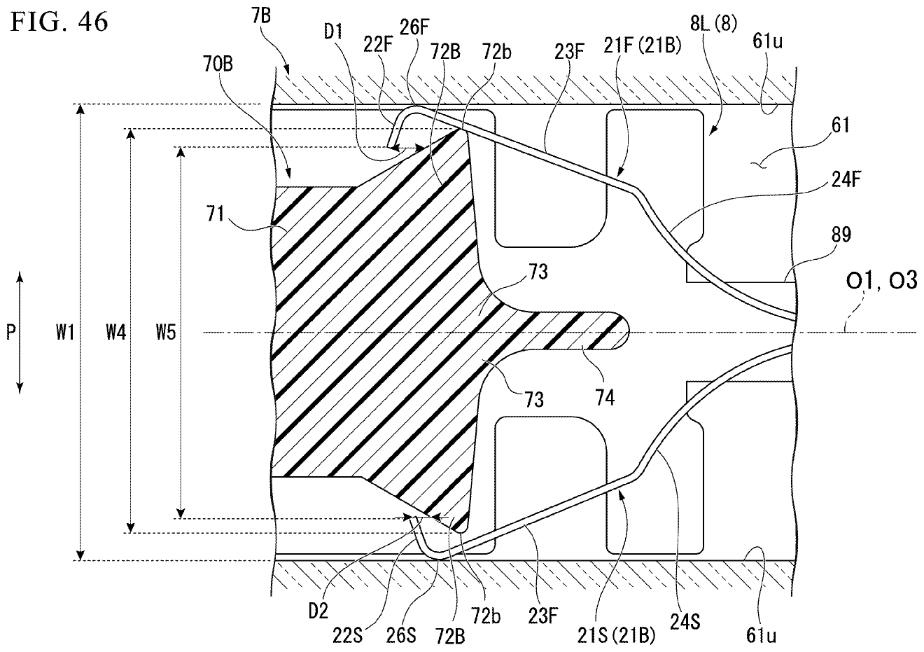

FIG. 46 is a diagram showing an intermediate layer member 70B of the restricting member 7B.

The restricting member 7B includes the intermediate layer member 70B and a guide member 8. The intermediate layer member 70B includes a distal end portion 71, a protruding portion 72B, a tapered portion 73, and a proximal end portion 74. The distal end portion 71, the protruding portion 72B, the tapered portion 73, and the proximal end portion 74 are arranged from the distal end to the proximal end along the central axis O3 direction of the restricting member 7B.

The protruding portion 72B is a member protruding in a protruding direction P substantially perpendicular to the central axis O3. The protruding portions 72B are provided on both sides of the central axis O3. The opening/closing direction P of the pair of arms 21 arranged on both sides of the protruding portion 72B substantially coincides with the protruding direction P of the protruding portion 72B. The first arm 21F and the second arm 21S are formed in an asymmetrical shape with respect to the central axis O1. Therefore, the first arm 21F and the second arm 21S, which are arranged on both sides of the protruding portion 72, are arranged at positions asymmetrical with respect to the central axis O3.

The intermediate layer member 70B is sandwiched between the upper layer guide member 8U and the lower layer guide member 8L in the height direction H, like the intermediate layer member 70 of the first embodiment.

The restricting member 7B is stored in the storage area 6S while being arranged inside the pair of arms 21B (on the closing side in the opening/closing direction P). When the clip unit 1B is stored in the storage area 6S together with the restricting member 7B, the restricting member 7B is arranged between the first arm 21F and the second arm 21S, and the distal ends of the claws (first claw 22F, second claw 22F) of the pair of arms 21B are separated from the restricting member 7B. Therefore, when assembling the cartridge system 100B, it is possible to prevent the distal ends of the claws of the pair of arms 21B from biting into the restricting member 7B.

When the clip unit 1B is stored in the storage area 6S together with the restricting member 7B, the shortest distance D1 between the distal end of the first claw 22F of the first arm 21F and the restricting member 7B is longer than the shortest distance D2 between the distal end of the second claw 22S of the second arm 21S and the restricting member 7B.

When the clip unit 1B is stored in the storage area 6S together with the restricting member 7B, the bending portions (the first bending portion 26F and the second bending portion 26S) of the pair of arms 21B are in contact with the inner surface 61u of the first area 61 of the case main body 60 in the opening/closing direction P. Further, when the clip unit 1B is stored in the storage area 6S together with the restricting member 7B, the first bending portion 26F and the second bending portion 26S are separated from the restricting member 7B.

In the protruding portion 72B, the portion protruding most in the protruding direction P from the central axis O3 is the maximum protruding point 72b. When the clip unit 1B is stored in the storage area 6S together with the restricting member 7B, the claws (first claw 22F, second claw 22S) and bending portions (first bending portion 26F and second bending portion 26S) of the pair of arms 21B are arranged on the distal end side of the maximum protruding point 72b.

The length W4 between the maximum protruding points 72b of the protruding portion 72B is slightly smaller than the length W1 of the first area 61 in the width direction W. Further, in the protruding direction P, the length W4 between the maximum protruding points 72b of the protruding portion 72B is longer than the length W5 between the distal ends of the claws (first claw 22F, second claw 22S) of the pair of arms 21B in the clip unit 1 stored in the storage area 6S. Therefore, when the pair of arms 21B are pulled toward the proximal end side in the first area 61, the pair of arms 21B do not come off the restricting member 7B, and the restricting member 7B remains arranged inside the pair of arms 21.

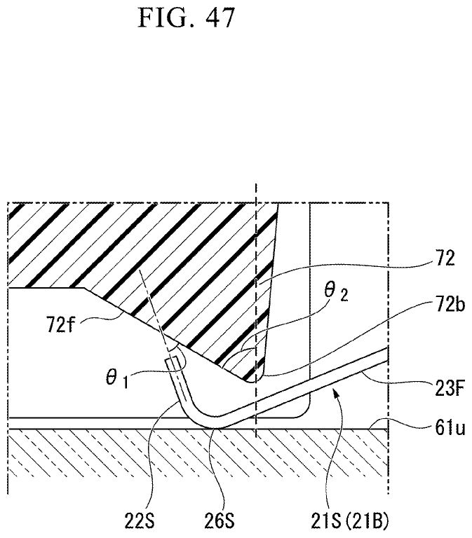

FIG. 47 is an enlarged view of the distal end of the second arm 21S.

When the clip unit 1B is stored in the storage area 6S together with the restricting member 7B, an angle θ1 formed by a facing surface 72f facing the distal end of the first claw 22F of the second arm 21S in the restricting member 7B and the direction in which the distal end of the second claw 22S of the second arm 21S is directed is greater than 0 degrees.

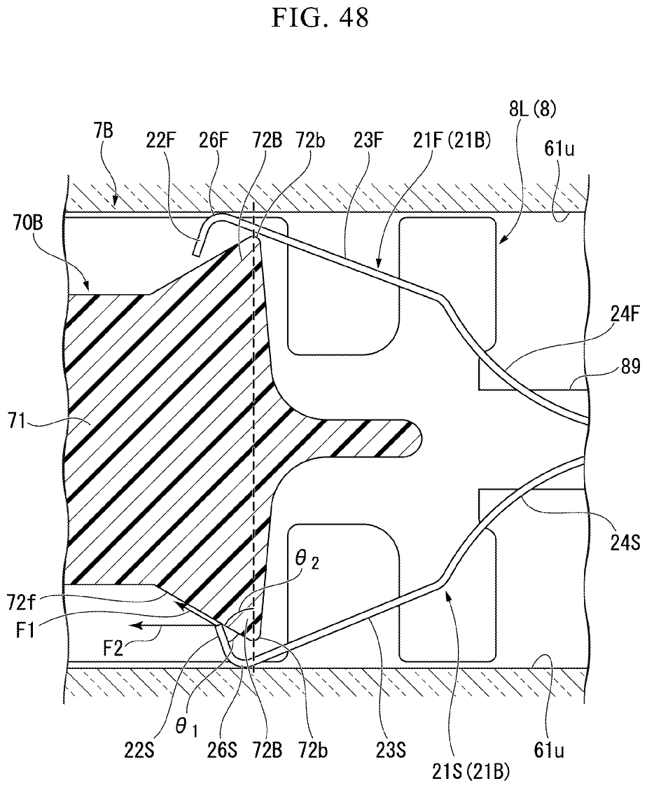

FIG. 48 is a diagram showing a pair of arms 21B pulled to the proximal end side.

When the pair of arms 21B are pulled to the proximal end side in the first area 61, the second claw 22S of the second arm 21S contacts the facing surface 72f of the intermediate layer member 70B. Since the shortest distance D1 is longer than the shortest distance D2, when the second claw 22S of the second arm 21S contacts the intermediate layer member 70B, the first claw 22F of the first arm 21F does not come into contact with the intermediate layer member 70B.

The greater the angle θ2, the smaller the frictional force F1 generated between the second claw 22S and the facing surface 72f when the second claw 22S contacts the facing surface 72f (see Equation 1). Here, μ is the coefficient of friction, and F2 is the reaction force generated when the restricting member 7B is pulled.

### [Number 1]

F1=μ·F2·cos θ₂ (Equation 1)

By increasing the angle θ2 to reduce the frictional force F1, when the restricting member 7B is pulled to the second area 62 as shown in FIG. 23, it is possible to prevent the pair of arms 21 from being caught by the intermediate layer member 70B and becoming difficult to open due to the frictional force F1. At this time, since the first claw 22F of the first arm 21F is not in contact with the intermediate layer member 70B, no frictional force is generated between the first claw 22F and the intermediate layer member 70B.

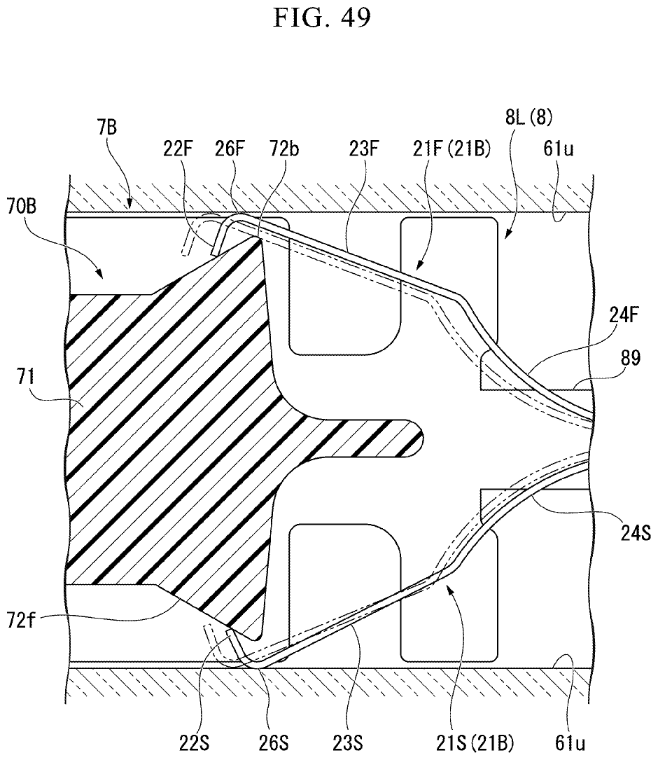

FIG. 49 is a diagram showing a pair of arms 21B whose distal ends are in contact with the restricting member 7B.

When the clip unit 1B is loaded into the clip introduction device 200, there is a case in which the pair of arms 21B are deformed as shown in FIG. 49 and both distal ends of the claws of the pair of arms 21B come into contact with the intermediate layer member 70B of the restricting member 7B. Even in this case, if the angle θ2 is large, the frictional force generated between the distal ends of the claws of the pair of arms 21B and the intermediate layer member 70B will be small.

According to the cartridge system 100B of the present embodiment, when the pair of arms 21B are pulled in order to load the clip unit 1 stored in the cartridge 5B into the clip introduction device 200, it is possible to prevent the distal ends of the claws (the first claw 22F and the second claw 22S) of the pair of arms 21B from being caught in the intermediate layer member 70B and becoming difficult to open.

As described above, the second embodiment of the present disclosure has been described in detail with reference to the drawings, but the specific configuration is not limited to this embodiment, and design changes and the like are also included within the scope of the present disclosure. Also, the constituent elements shown in the above-described embodiment and modifications shown below can be combined as appropriate.

### Modification 2-1

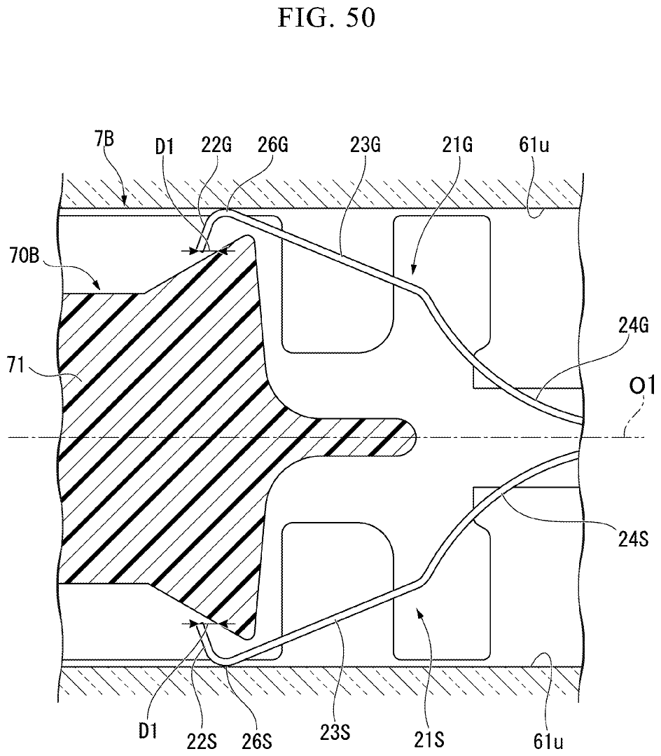

In the above embodiment, the pair of arms 21, 21B are formed in an asymmetrical shape with respect to the central axis O1. However, as shown in FIG. 50, the pair of arms may be formed in a shape symmetrical with respect to the central axis O1. The pair of arms shown in FIG. 50 includes a first arm 21G and a second arm 21S. The first arm 21G and the second arm 21S are arranged symmetrically with respect to the central axis O1 of the clip unit 1 and formed in a shape symmetrical with respect to the central axis O1. In this case, the frictional force generated between the distal ends of the claws of the pair of arms (the first claw 22G and the second claw 22S) and the intermediate layer member 70B is reduced, and it is possible to prevent the distal ends of the claws of the pair of arms from being caught in the intermediate layer member 70B and becoming difficult to open.

### Industrial Applicability

The present disclosure can be applied to cartridges and the like that store clip units.

Example 1. A cartridge system, comprising:
a clip unit including a first arm and a second arm, the second arm is shorter than the first arm;
a case including a storage area storing the clip unit along a longitudinal direction; and
a block configured to be stored in the storage area together with the clip unit, wherein when the clip unit is stored in the storage area together with the block, the block is arranged between the first arm and the second arm.

Example 2. The cartridge system according to Example 1, wherein the first arm including a first claw provided at a distal end of the first arm and a first arm main body supporting the first claw, wherein the second arm including a second claw provided at a distal end of the second arm and a second arm main body supporting the second claw, and wherein, when the clip unit is stored in the storage area together with the block, a distal end of the first claw is separated from the block, and a distal end of the second arm is separated from the block.

Example 3. The cartridge system according to Example 2, wherein the second arm including a second bending portion bending inward , the a second bending portion is located between second arm main body and the second claw, and wherein the clip unit is stored in the storage area of the case in a state that the second bending portion is in contact with the case.

Example 4. The cartridge system according to Example 3, wherein the clip unit is stored in the storage area of the case in a state in which the second bending portion is separated from the block.

Example 5. The cartridge system according to claim Example 2, wherein the block including a surface of the block facing the distal end of the second claw, the clip unit is stored in the storage area of the case in a state in which an angle is greater than 0 degree, the angle is formed by the surface and a direction in which the distal end of the second claw faces.

What is claimed is:

1. A cartridge system, comprising:
a clip unit including an arm configured to open and close; and
a case including a storage area , the storage area configured so that the clip unit moves along a longitudinal direction, wherein the case includes an opening through which at least part of the arm is exposed when the clip unit stored in the storage area is positioned at a predetermined position.

2. The cartridge system according to claim 1, further comprising:
a restricting member configured to be stored in the storage area while being grasped by the arm, wherein the predetermined position is a distal end side position where the restricting member grasped by the arm of the clip unit stored in the storage area engages with the case on a distal end side in the longitudinal direction.

3. The cartridge system according to claim 1, wherein the predetermined position is a proximal side position where the clip unit stored in the storage area engages with the case on a proximal side in the longitudinal direction.

4. The cartridge system according to claim 1, wherein the opening is formed at a position where at least a portion of the arm of the clip unit placed at any position in the storage area is exposed.

5. The cartridge system according to claim 1, wherein the opening penetrates the case in a height direction that intersects with the longitudinal direction and the opening communicates with the storage area.

6. The cartridge system according to claim 5, wherein the opening including an upper opening provided on a first side in the height direction and a lower opening provided on a second side in the height direction, and wherein, when viewed from the height direction, the upper opening overlaps at least a part of the lower opening.

7. The cartridge system according to claim 1, further comprising:
a restricting member configured to be stored in the storage area while being grasped by the arm, wherein the restricting member includes a through-space penetrating in a height direction that intersects with the longitudinal direction, and
the through-space overlaps at least a portion of the arm grasping the restricting member when viewed from the height direction, and wherein, when the clip unit stored in the storage area is positioned at the predetermined position, the opening overlaps the through-space when viewed from the height direction.

8. The cartridge system according to claim 7, wherein the through-space is divided into an outer through-space and an inside through-space, the outer through-space is located outside the arm in a width direction perpendicular to the height direction, the inside through-space is located inside the arm in the width direction.

9. The cartridge system according to claim 1, wherein the arm includes a curve portion at a position overlapping the opening when viewed from a height direction intersecting the longitudinal direction when the clip unit is stored in the storage area.

10. A cartridge system, comprising:
a clip unit including an arm configured to open and close;
a case including a storage area , the storage area configured so that the clip unit moves along a longitudinal direction; and
a thread including a first end;
wherein the first end is tied to the arm.

11. The cartridge system according to claim 10, wherein the case including an opening, the thread including second end, a second end passes through the opening, and the second end is arranged outside the case.

12. The cartridge system according to claim 10, further comprising:
a package housing the case storing the clip unit.

13. A cartridge system, comprising:
a clip unit including a plurality of arms configured to open and close;
a case including a storage area storing the clip unit along a longitudinal direction; and
a block configured to be stored in the storage area together with the clip unit, wherein each arm of the plurality of arms includes a claw provided at a distal end of each arm, an arm body supporting the claw, and a bending portion that connects the arm body and the claw and bends inward, and wherein, when the clip unit is stored in the storage area together with the block, the block is arranged inside the plurality of arms, and the bending portion is arranged away from the block.

14. The cartridge system according to claim 13, wherein the clip unit is stored in the storage area of the case in a state in which the bending portion is in contact with the case.

15. The cartridge system according to claim 13, wherein the block including a facing surface facing a distal end of the claw, the clip unit is stored in the storage area of the case in a state in which an angle is greater than 0 degrees, the angle is formed by the facing surface and a direction in which the distal end of the claw faces.

16. The cartridge system according to claim 13, wherein the block including protruding portions protruding on both sides in an opening/closing direction of the plurality of arms, and wherein, in the opening/closing direction, a length between maximum protruding points of the protruding portions is longer than a length between the distal end of the claw for each of the plurality of arms in the clip unit stored in the storage area.

17. The cartridge system according to claim 16, wherein the clip unit is stored in the storage area in a state in which the bending portion is arranged on a distal end side of a particular maximum protruding point of a particular protruding portion.

18. The cartridge system according to claim 13, wherein the block is stored in the storage area of the case so as to be movable in the storage area in the longitudinal direction together with the clip unit.

19. The cartridge system according to claim 13, wherein the block is stored in the storage area of the case while being separably engaged with the clip unit.

20. The cartridge system according to claim 13, wherein the plurality of arms have a first arm and a second arm, the second arm is shorter than the first arm, and wherein, when the clip unit is stored in the storage area together with the block, a distance between the distal end of the claw of the first arm and the block is longer than a distance between the distal end of the claw of the second arm and the block.
